(19) United States
(12) Patent Application Publication
(10) Pub. No.: US 2026/0242411 A1
(43) Pub. Date: Aug. 20, 2026

(54) FLUORESCENT LIPID COMPOUNDS

(71) Applicant: Florida Atlantic University Research Corporation, Boca Raton, FL (US)

(72) Inventors: Maciej STAWIKOWSKI, Boca Raton, FL (US); Qi ZHANG, Boca Raton, FL (US)

(21) Appl. No.: 19/164,923

(22) PCT Filed: Jan. 12, 2024
(86) PCT No.: PCT/US2024/011374

§ 371 Date: Sep. 12, 2025

Related U.S. Application Data
(60) Provisional application No. 63/490,354, filed on Mar. 15, 2023.

(51) Int. Cl. C07J 43/00; A61K 49/00; G01N 21/64

(52) U.S. Cl. CPC C07J 43/003; A61K 49/0021; A61K 49/0028; G01N 21/6428; G01N 2021/6439

(57) ABSTRACT

Disclosed herein are compounds useful for imaging of lipid systems. Disclosed herein are fluorescent probes which closely mimic the structure and functions of natural cholesterol and other lipids.

### CROSS-REFERENCE TO RELATED APPLICATION

This application claims the benefit of U.S. Provisional Application 63/490,354, filed Mar. 15, 2023, the contents of which is hereby incorporated in its entirety.

### STATEMENT OF GOVERNMENT SUPPORT

This invention was made with government support under R15GM147912-01 awarded by the National Institutes of Health. The government has certain rights in the invention.

### FIELD OF THE INVENTION

The invention is directed to molecular probes and tools for the study of cellular systems. In some implementations, the probes include cholesterol and other lipid derivatives.

### BACKGROUND

The plasma membrane (PM) not only protects cytoplasmic content but also mediate cells' interaction with the environment through numerous transmembrane signaling pathways. As one of the major membrane lipids, cholesterol (Chol) is biophysically and functionally critical to cell membranes. Owning to its unique structure, cholesterol play a decisive role in organizing the lipid bilayer and membrane proteins. In particular, cholesterol content determines the fluidity of lipid membrane and thus the mobility as well as the stability of membrane proteins. Hence, membrane-embedded cholesterol (mChol) is tightly controlled by mechanisms which is largely unexplored.

The human brain is probably the most Chol-rich organ as it is only about 1/20 of body weight but contains over ⅕ of total body Chol/In addition to myelin sheath, the neuronal PM is also enriched with cholesterol (~30-35%), more than most cells in the body have. In developing neurons, cholesterol is required for neuronal differentiation, neurite growth and synaptogenesis. In mature neurons, cholesterol is essential for ion channel activity, synaptic vesicle (SV) exo-/endocytosis, and receptor activity. So, cholesterol reduction can cause global synaptic dysfunction, neurite disintegration, and eventually neuronal loss, which is reminiscent to Alzheimer's disease (AD).

Due to the separation by the blood-brain barrier, brain, cholesterol is regulated independent of serum Chol. Generally, astrocytes synthesize cholesterol and supply it to neurons in the form of high-density lipoprotein (HDL) and low-density lipoprotein (LDL) made of ApoE. Like most cells in the body, neurons take up HDL/LDL by lipoprotein receptors like LRP1 via receptor-mediated endocytosis. Recent report suggests that the “HDL-like” particles are secreted mostly by younger astrocytes while the “LDL-like” particles are secreted by older ones, suggesting that aging affects brain cholesterol metabolism. The internalized HDL and LDL enters endosomes and endosome sorting pathway separates cholesterol and other lipids from ApoE proteins. In endosomes and lysosomes, cholesteryl ester (CE) carried by HDL/LDL are digested by mainly by lysosomal acid lipase (LAL), and the liberated cholesterol is inserted into the membrane of late endosome/lysosome by coordinated actions of NPC1, NPC2, and lysosome-associated membrane glycoprotein 2. Subsequently, cholesterol is distributed to needed membrane compartments. Generally, cholesterol transportation is mediated by membrane fusion and fission between the organelles, through so called vesicular transport. From surface to lysosomes, cholesterol experiences a significant pH drop.

While ~80% cholesterol is acquired and transported via vesicular transport, the recently reported nonvesicular transport (i.e., protein-assisted) appears to play a significant role in cholesterol trafficking between organelles. Nonvesicular trafficking requires membrane contact sites (MCSs) and sterol transport proteins (STPs). STPs is part of lipid transport proteins (LTPs), which provide hydrophobic pockets to shield cholesterol from the aqueous phase and thus guarantee proper transfer.

Several proteins, including members of the steroidogenic acute regulatory protein (StAR)-related domain lipid transfer (START) family and oxysterol-binding protein (OSBP)-related proteins (ORPs), are thought to play a role in nonvesicular cholesterol transport within the cytosol's aqueous environment and between organelles and the PM.

Given the vital role cholesterol and other lipids play in cell function, the remains a need for improved methods and systems for imaging, studying, and manipulating biological systems that make use of lipids. Existing fluorescent probes for studying cholesterol fall roughly in two categories, sensors selectively binding to cholesterol and analog probes. The commonly used fluorescent Chol-sensors are Filipin and Perfringolysin O (particularly its domain 4, D4). Filipin is an intrinsically fluorescent polyene antibiotic that binds to non-esterified cholesterol. However, it has a few drawbacks including nonspecific binding to other lipids and permeabilizing cell membranes, making it unsuitable for live-cell application. Moreover, Filipin requires UV excitation and emits weak blue fluorescence, leading to high cytotoxicity (because of UV), poor fluorescence detection (since most photosensors have low quantum efficiency for blue light), and inadequate spatiotemporal resolution (due to its weak signal-to-noise ratio). Perfringolysin O is a toxin selectively binding to cholesterol with its D4, and thus genetically recombinant D4 has been conjugated with fluorophores to serve as a cholesterol sensor. Recombinant EGFP/mCherry-D4s were recently used to visualize cholesterol in the exofacial leaflets of the PM in the living cells. However, D4-like labels suffer from its bulky size and interference by cytosolic proteins and potentially disrupts endogenous Chol.

Cholesterol interactions with surroundings are complex and can occur through hydrophobic (sterol) part of the molecule, —OH interaction, or both. This must be taken into consideration when designing a cholesterol probe. Due to the complexity of cholesterol interactions, both property criteria (chemistry and fluorescent tag) are mutually exclusive to some extent. So, different probes biased toward one or the other criteria depend on the applications. Available fluorescent cholesterol analogs can be divided into intrinsically fluorescent sterols and extrinsic or tagged sterol. Natural fluorescent sterols have several conjugated double bonds, either in the steroid ring system (e.g. dehydroergosterol, DHE) or in the fatty acyl moiety in the case of an ester. The disadvantage of DHE is its poor fluorescence, very similar to Filipin. The environmental sensitivity of DHE is low since the molecular dipole does not change greatly during electronic transition from the ground S₀, state to the excited S₁ state. Synthetic sterol probes often have a fluorophores attached to either the steroid backbone or the fatty acyl chain in the case of a ester analog. The two most frequently fluorophores used to label cholesterol are NBD (nitrobenzoxadiazole) and BODIPY (boron dipyrromethene core). The NBD is smaller than bulky BODIPY group and NBD unlike the BODIPY is an environmentally-sensitive fluorophore. On the other hand BODIPY is much brighter. Modification of Chol's hydrophobic tail with NBD resulted in 25-NBD-NBD and 22-NBD-cholesterol conjugates used to study cholesterol mimicking. It is recognized that 22-NBD-cholesterol does not behave like cholesterol as its partition in Lo/Ld membranes is opposite to that of Chol. Nevertheless, 22-NBD-cholesterol and DHE but not BODIPY-cholesterol have been used to study sterol import into yeast cells by ABC transporters. The BODIPY-cholesterol (a.k.a TF-Chol, Bdp-Chol, BChol) was created to better mimic cholesterol since BODIPY is electrically neutral and fluorescently brighter. It has been well recognized and widely used in cellular assays including live-cell tests like labeling different membrane phases. However, its intracellular trafficking differs from cholesterol and molecular dynamics (MD) simulations of B cholesterol indicate that it has a higher molecular tilt compared to Chol. Interestingly, studies using B cholesterol showed that part of the sterol transport between the PM and the endocytic recycling compartment is nonvesicular transport.

There remains a need for improved compounds for lipid imaging. There remains a need for improved probes and sensors with membrane and pH selectivity. There remains a need for improved probes and sensors that more closely mimic native cholesterol and other lipids.

### BRIEF DESCRIPTION OF THE FIGURES

FIG. 1 depicts structures and properties (i.e., membrane- and pH-sensitivities) of developed model probes CND2, CND3 and CND5.

FIG. 2 depicts a sample image shows near complete overlap between CND2 and FM4-64 at axon terminals, suggesting CND2 enters acidic synaptic vesicles. B, CND2 and FM4-64 co-imaging in the same neurons with 10-Hz 120-s electrical field stimulations and final pH manipulations (50-mM NH4Cl and pH5.5 for the baseline and total CND2 fluorescence.

FIG. 3 depicts the behavior of CND probes in model membrane (25% CHOL, 75% POPC) obtained from 100 ns MD simulations. Top panel: Probe conformation and position in the membrane. Green—phosphorus atoms; blue—cholesterol. Bottom panel: immersion of CND2 and CND3 in the membrane. The —OH group in CND-3 linker (green line) immerses in the membrane just like native cholesterol (blue line).

FIG. 4 depicts additional compounds of invention, and a general scheme for their synthesis.

FIG. 5 depicts sample colocalization studies in cultured astrocytes. CND2 (green) and markers: Nile Red (red) and LysoView (blue) 24 h after 1 h exposure with the probe at 1 uM and subsequent wash. White line intensity profile is plotted below. The difference in probe colocalization/uptake is evident.

FIG. 6 depicts images of the staining of hippocampal cell culture 24 h after 1 h exposure to CND2 probe shown in orthogonal view projection. Neurons and astrocytes differ in the amount and localization of uptaken CND2 probe (green puncta) suggesting difference in cholesterol distribution and regulation. Nile Red staining shown in red.

FIG. 7A depicts absorbance (left columns) and fluorescence (right columns) of CND1 obtained in various organic solvents, demonstrating solvatochromic effect (environmental sensitivity).

FIG. 7B depicts absorbance (left columns) and fluorescence (right columns) of CND2 obtained in various organic solvents, demonstrating solvatochromic effect (environmental sensitivity).

FIG. 7C depicts absorbance (left columns) and fluorescence (right columns) of CND3 obtained in various organic solvents, demonstrating solvatochromic effect (environmental sensitivity).

FIG. 7D depicts absorbance (left columns) and fluorescence (right columns) of CND4 obtained in various organic solvents, demonstrating solvatochromic effect (environmental sensitivity).

FIG. 7E depicts absorbance (left columns) and fluorescence (right columns) of CND5 obtained in various organic solvents, demonstrating solvatochromic effect (environmental sensitivity).

FIG. 7F depicts absorbance (left columns) and fluorescence (right columns) of CND6 obtained in various organic solvents, demonstrating solvatochromic effect (environmental sensitivity).

FIG. 7G depicts absorbance (left columns) and fluorescence (right columns) of CND7 obtained in various organic solvents, demonstrating solvatochromic effect (environmental sensitivity).

FIG. 7H depicts absorbance (left columns) and fluorescence (right columns) of CND8 obtained in various organic solvents, demonstrating solvatochromic effect (environmental sensitivity).

FIG. 7I depicts absorbance (left columns) and fluorescence (right columns) of CND9 obtained in various organic solvents, demonstrating solvatochromic effect (environmental sensitivity).

FIG. 7J depicts absorbance (left columns) and fluorescence (right columns) of CND10 obtained in various organic solvents, demonstrating solvatochromic effect (environmental sensitivity).

FIG. 8 depicts pH-dependent fluorescence dependence of CND2, CND3 and CND4 probes. Probes were dissolved at 1 μM in 1% solution of octyl glucoside micelles in phosphate buffer of different pH. Excitation wavelength—405 nm. The difference in fluorescence between pH 7 (cytosolic) and pH 5 (lysosomes) is measured to be 2.4, 2.7, and 2.1-fold for CND2, CND3, CND4 respectively.

FIG. 9A depicts the emission spectra of CND1, CND2, and CND3 at 1 μM in 1% octyl glucoside micelles as compared with water. FIG. 9B depicts the emission spectra of CND4, CND5, and CND6 at 1 μM in 1% octyl glucoside micelles as compared with water. FIG. 9C depicts the emission spectra of CND10, CND8, and CND9 at 1 μM in 1% octyl glucoside micelles as compared with water. This shows the potential for membrane sensing, but also reveals potential for probe aggregation in water. The fluorescence in water is attributed to aggregation induced emission (AIE) phenomenon.

FIG. 10 depicts GUVs formed by electroformation in 2:2:1 DOPC: SM: Chol with 0.1% mol of probe and Ld marker (DiD). Example of GUV containing CND probe (left). Analysis of lipid phase partitioning of CND Series (right). Data is compared to other existing cholesterol sensors published elsewhere (Sezgin E, et al. J Lipid Res. 2016 February; 57(2):299-309)

FIG. 11 depicts the determination of uptake and subcellular distribution of CND1 (FIG. 11B) and CND3 (FIG. 11C) probes as compared with TFC (BODIPY-Cholesterol) (FIG. 11A) probe. Live mouse astrocytes were incubated with fluorescent probes at 1 μM for 1 h. After that, excess of probes was washed away by replacing of culture media and imaged under confocal microscope at 1 h and 24 h after incubation. CND probes are found in endocytic vesicles (endosomes and lysosomes) and lipid droplets. CND1 after 24 also shows broader diffusion across all membranes (more intensity in the cytoplasmic area) similarly to TFC).

FIGS. 12A and 12B depict the determination of colocalization of CND probes. Probes were tested for organelle colocalization. Nile red dye was used as marker for lipid droplets, Lysoview650 marker was used for lysosomes. Cells were incubated with fluorescent probes at 1 uM and both markers for 1 h. After that, excess of probes was washed away by replacing of culture media and imaged under confocal microscope at 1 h and 24 h after incubation. Due to low specificity of LD marker and partial overlap with lysosomal marker, before determining of Pearson's colocalization coefficient (PCC) fluorescent images from LD and lysosome channels were subtracted from each other. The resulting LD and lysosomal images were then tested for colocalization with CND fluorescent channel. Pearson's coefficient was determined using EzColocalization plugin for ImageJ software.

FIG. 13 depicts the difference in CND probes colocalization between 24 h and 1 h data points for lysosomes (Lyso View marker) and lipid droplets (Nile red marker) in different types of cells. In both cell types CND2-4 tend to accumulate in lipid droplets. CND1 shifts colocalization to lysosomes in both cell types, but CND8 colocalization to lysosomes is only observed in mouse astrocytes.

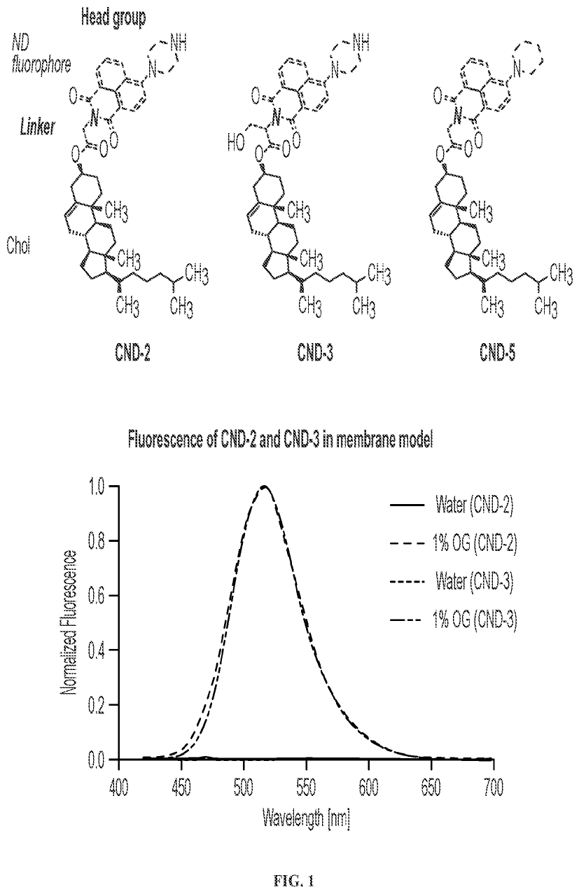

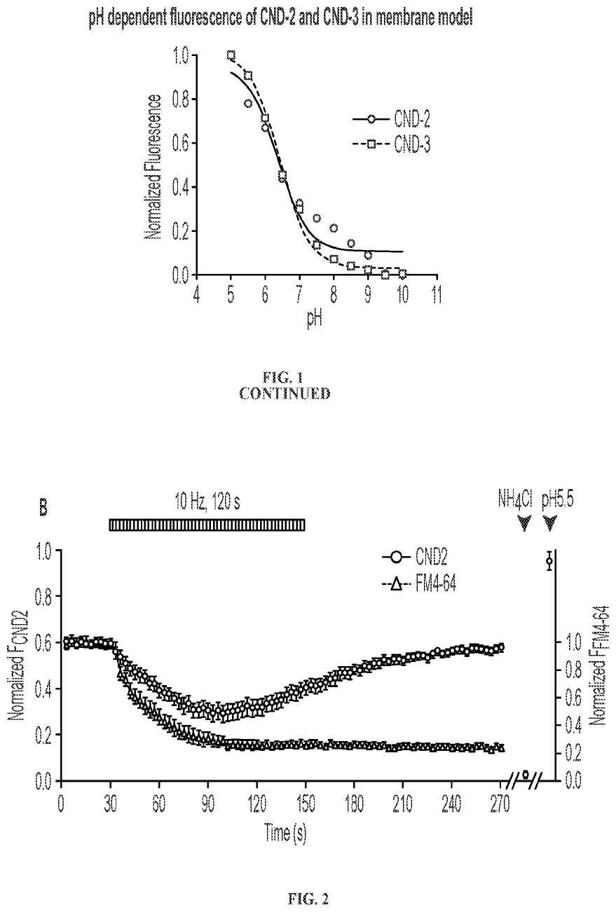

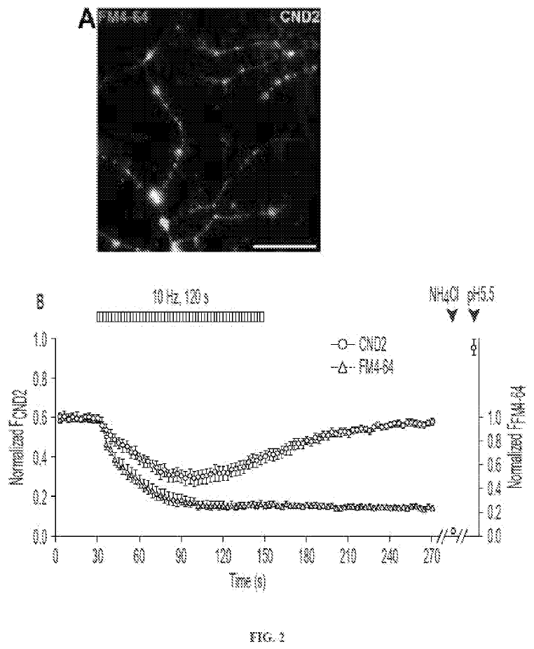

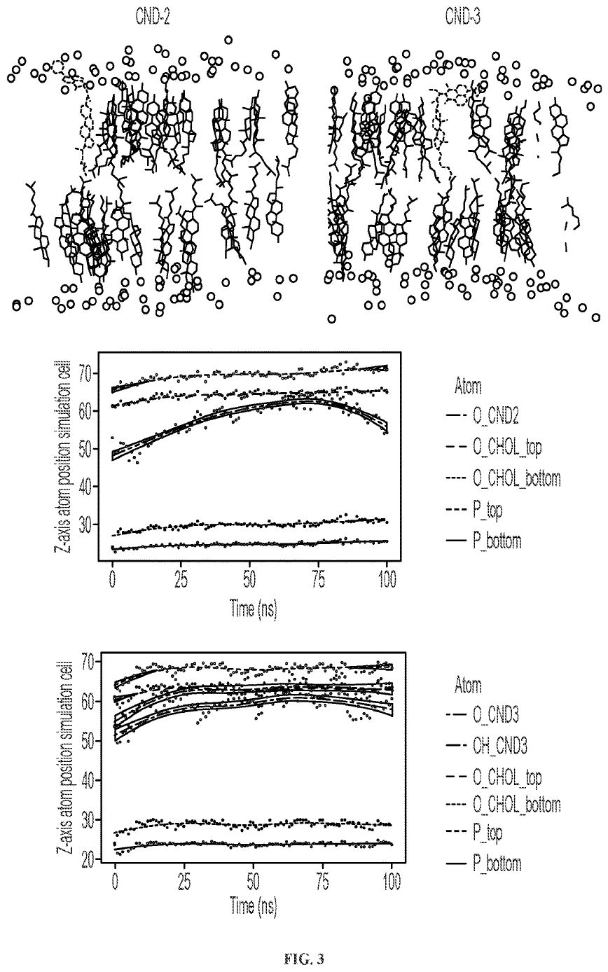

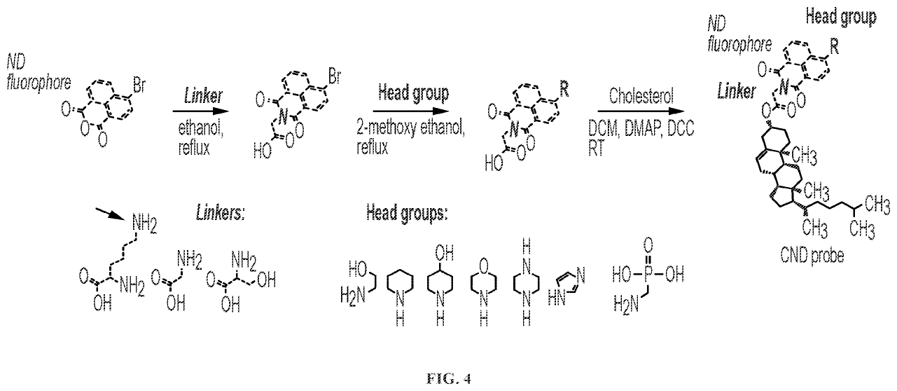

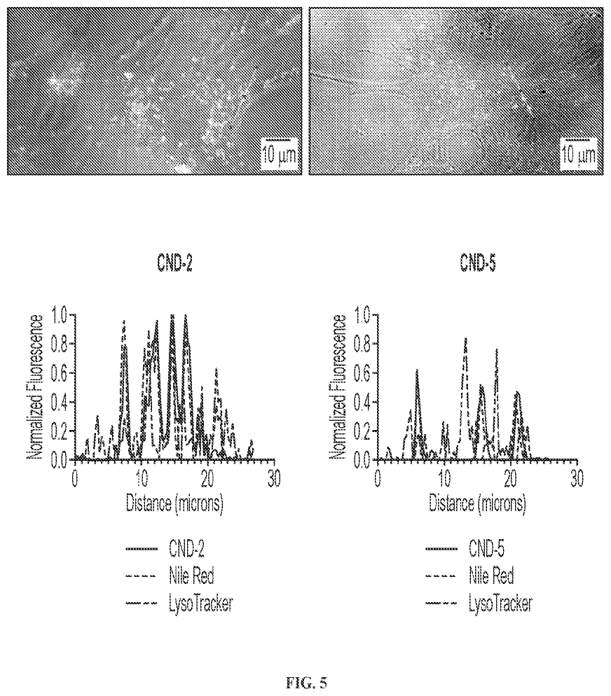

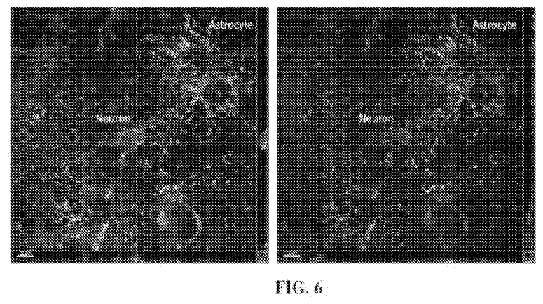

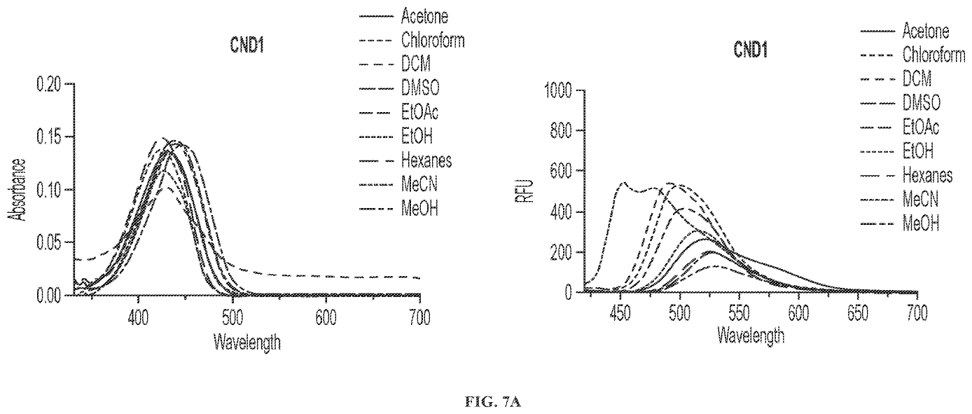

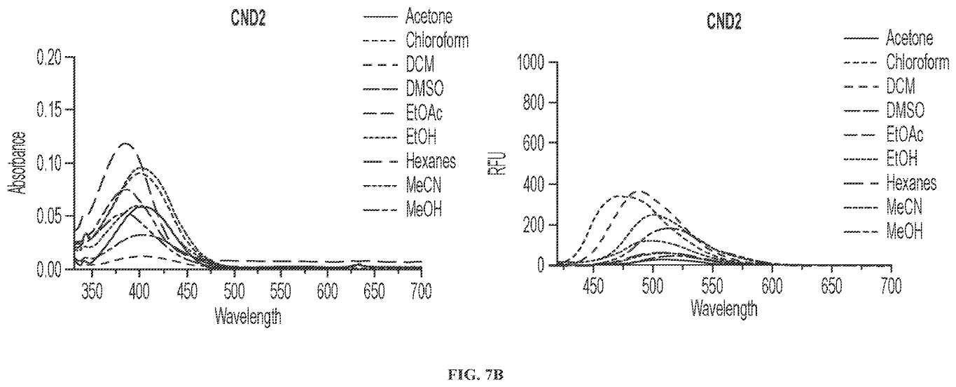

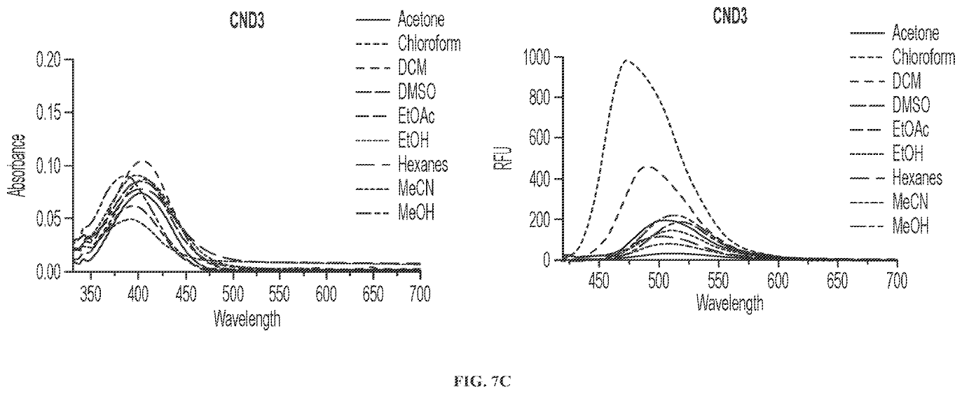

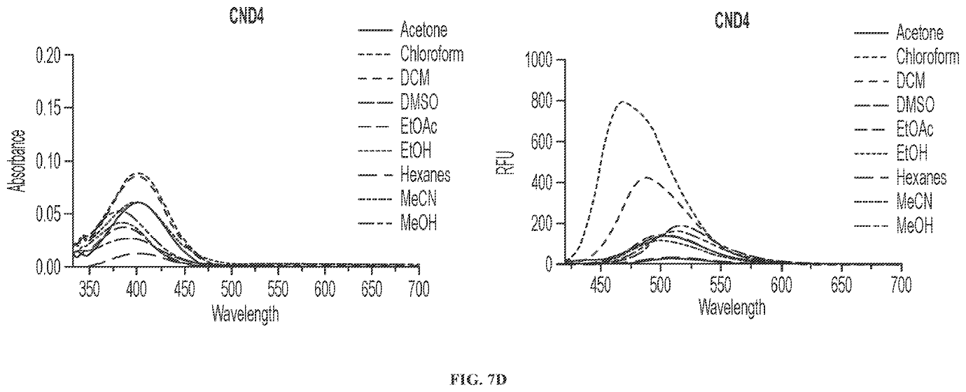

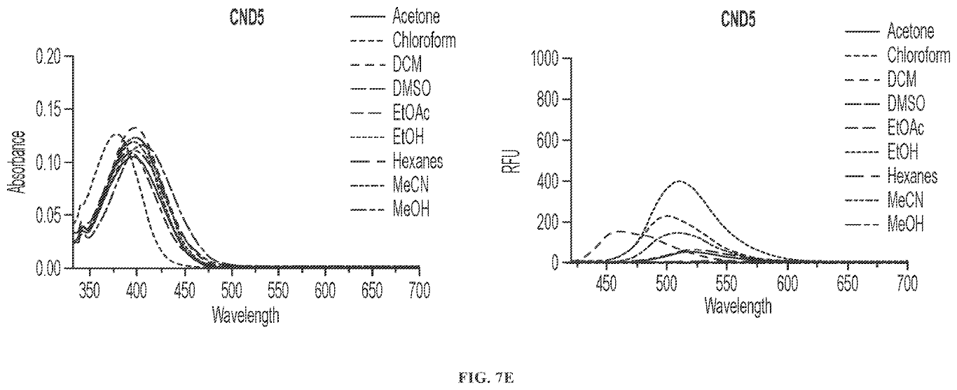

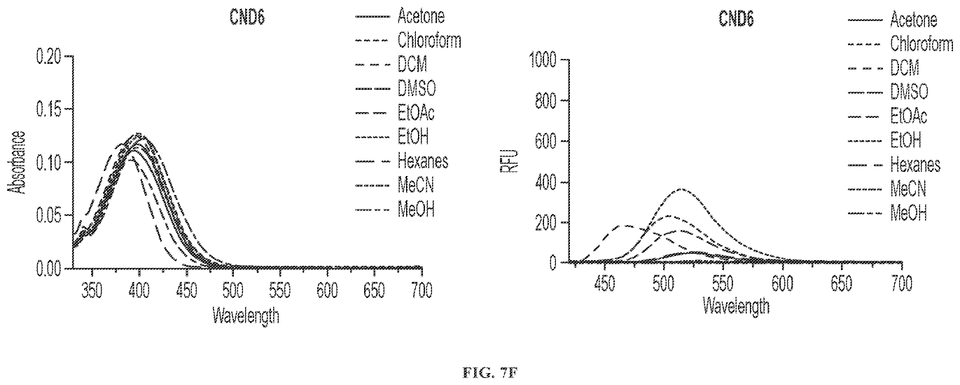

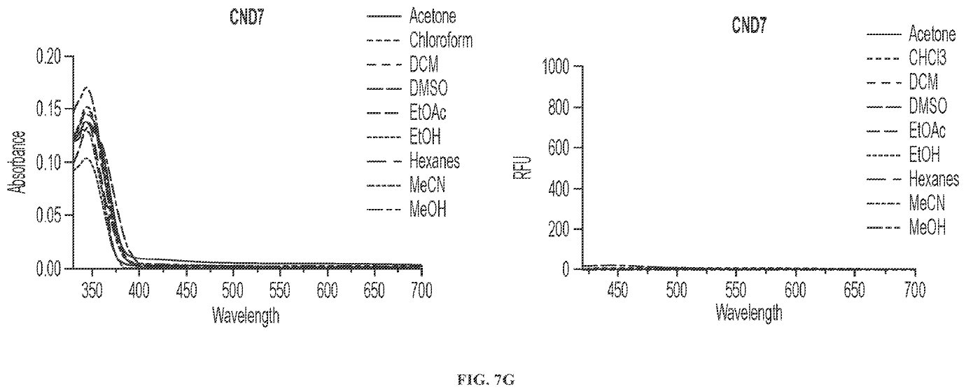

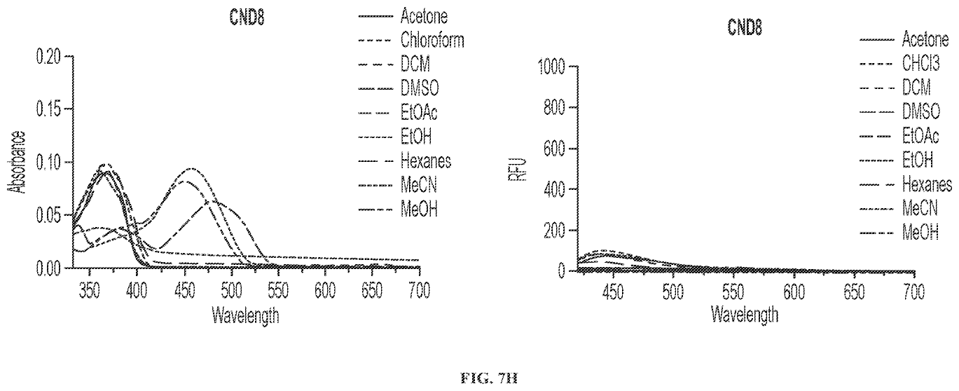

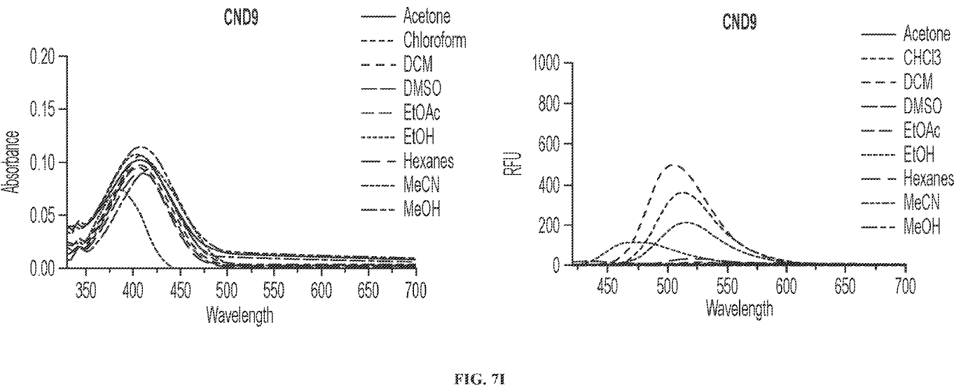

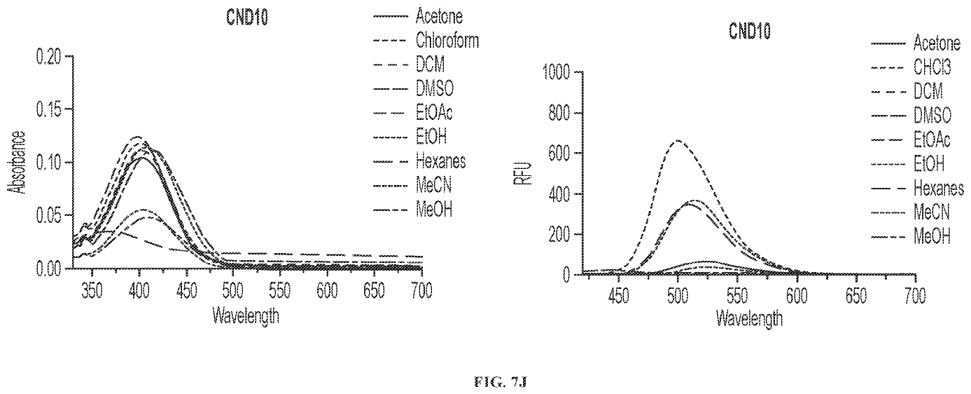

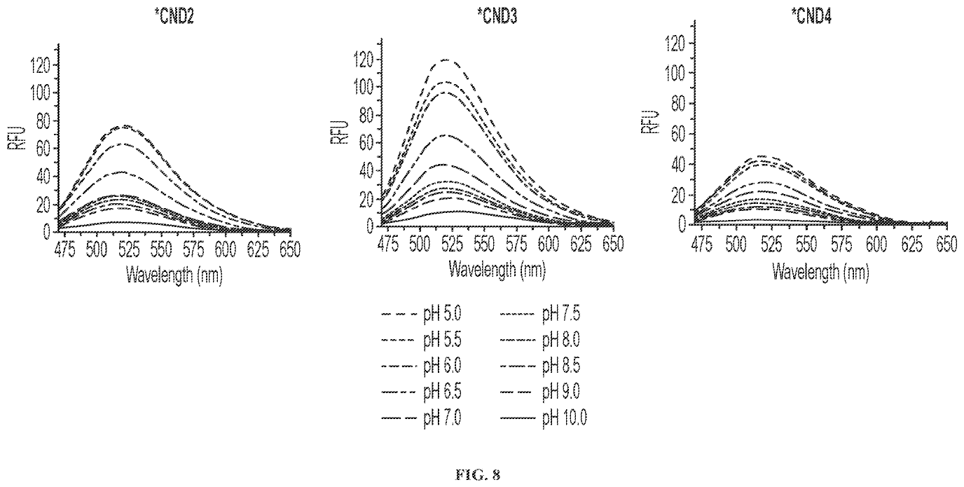

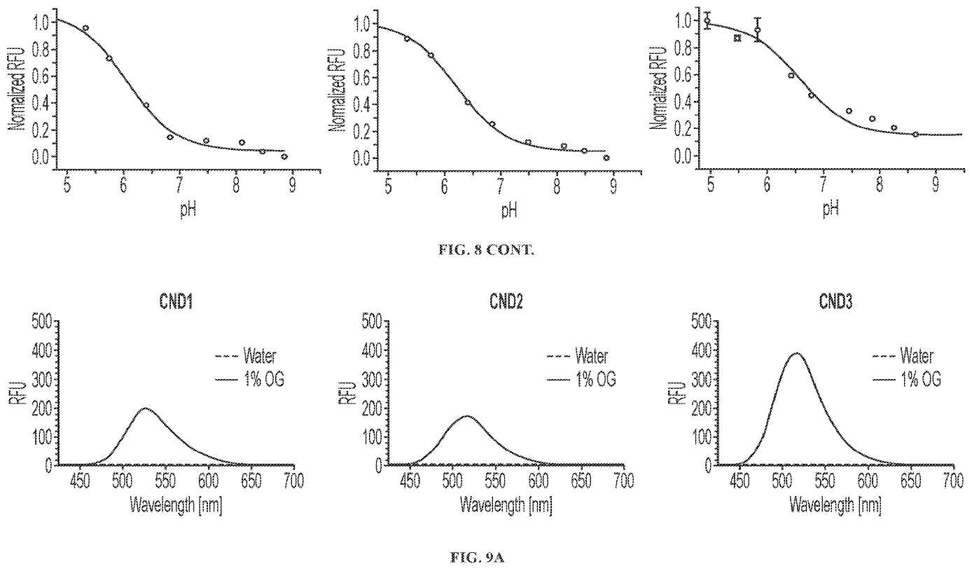

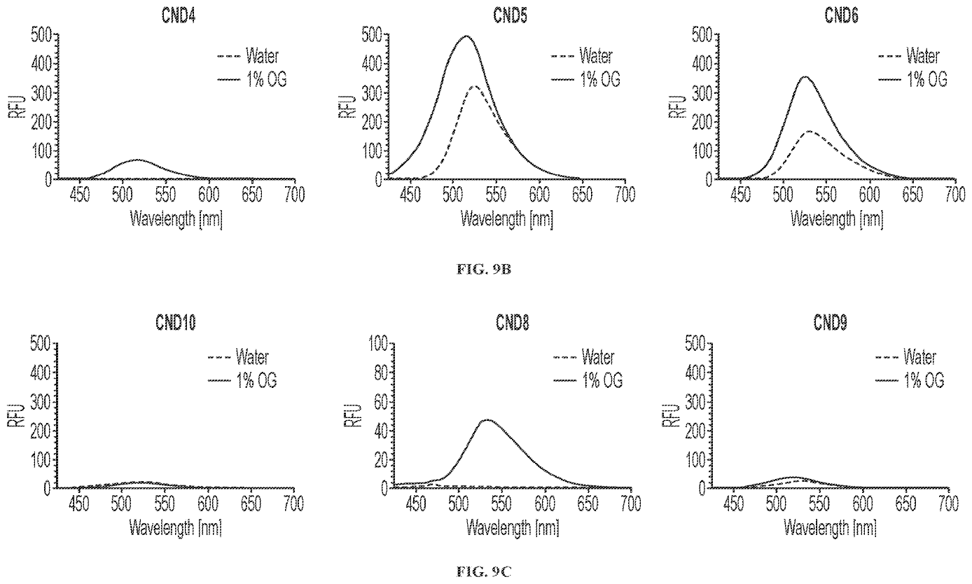

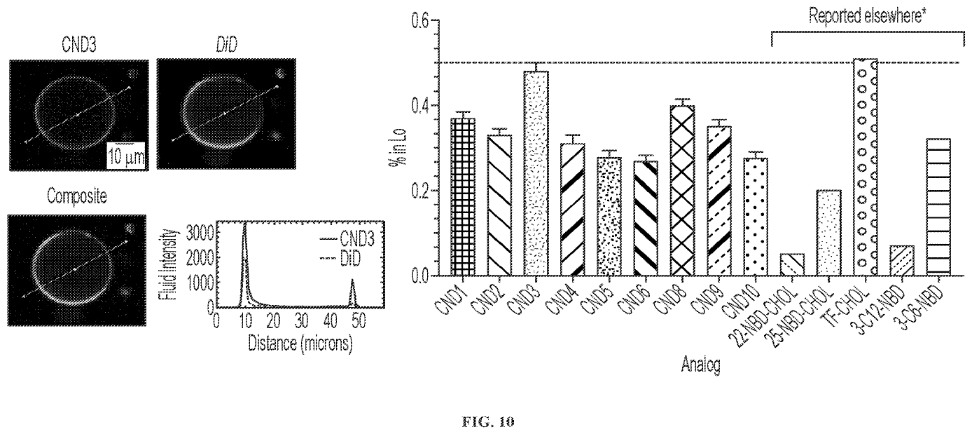

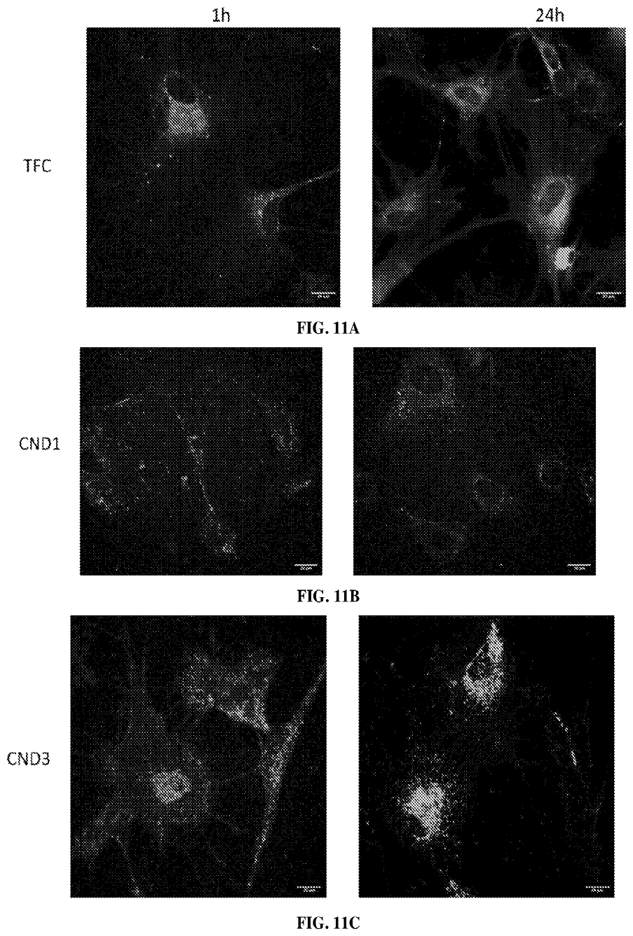

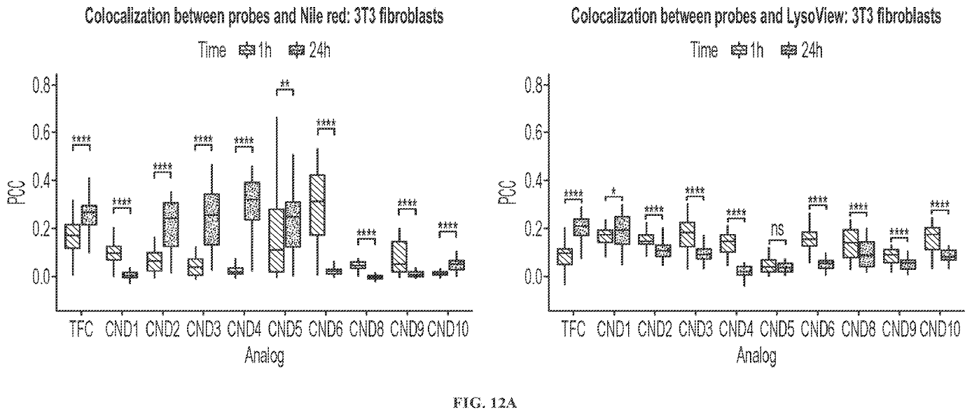

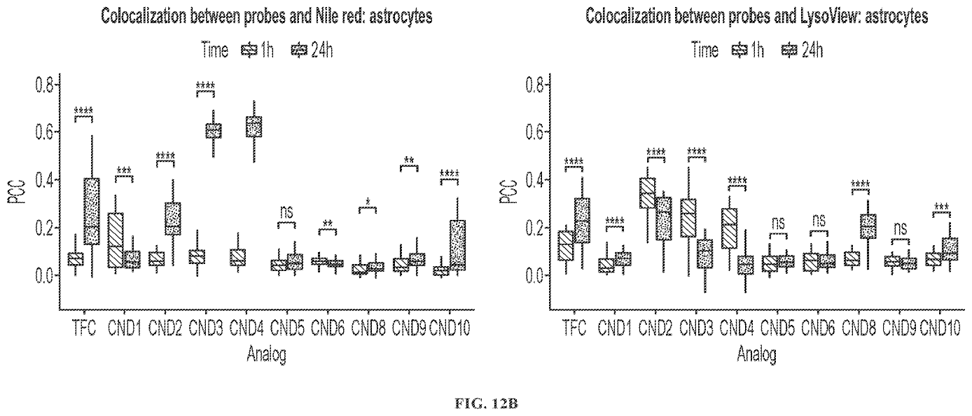

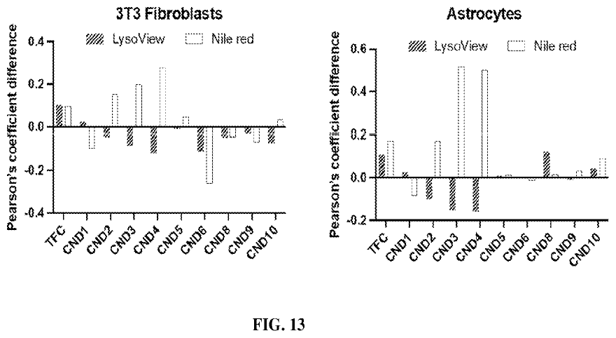

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of physiologically acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched hydrocarbon group having a specified range of carbon atoms (e.g., a “C₁₋₁₆ alkyl” can have from 1 to 16 carbon atoms). In some embodiments, an alkyl group has 1 to 9 carbon atoms (“C₁₋₉ alkyl”). An alkyl group can be saturated or unsaturated, i.e., an alkenyl or alkynyl group as defined herein. Unless specified to the contrary, an “alkyl” group includes both saturated alkyl groups and unsaturated alkyl groups.

In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, Bn).

The term “haloalkyl” is a substituted alkyl group, wherein one or more of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some

embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “hydroxyalkyl” is a substituted alkyl group, wherein one or more of the hydrogen atoms are independently replaced by a hydroxyl. In some embodiments, the hydroxyalkyl moiety has 1 to 8 carbon atoms (“C₁₋₈ hydroxyalkyl”). In some embodiments, the

hydroxyalkyl moiety has 1 to 6 carbon atoms (“C₁₋₆ hydroxyalkyl”). In some embodiments, the hydroxyalkyl moiety has 1 to 4 carbon atoms (“C₁₋₄ hydroxyalkyl”). In some embodiments, the hydroxyalkyl moiety has 1 to 3 carbon atoms (“C₁₋₃ hydroxyalkyl”). In some embodiments, the hydroxyalkyl moiety has 1 to 2 carbon atoms (“C₁₋₂ hydroxyalkyl”).

The term “alkoxy” refers to an alkyl group, as defined herein, appended through an oxygen atom. In some embodiments, the alkoxy moiety has 1 to 8 carbon atoms (“C₁₋₈ alkoxy”). In some embodiments, the alkoxy moiety has 1 to 6 carbon atoms (“C₁₋₆ alkoxy”). In some embodiments, the alkoxy moiety has 1 to 4 carbon atoms (“C₁₋₄ alkoxy”). In some embodiments, the alkoxy moiety has 1 to 3 carbon atoms (“C₁₋₃ alkoxy”). In some embodiments, the alkoxy moiety has 1 to 2 carbon atoms (“C₁₋₂ alkoxy”). Representative examples of alkoxy include, but are not limited to, methoxy, ethoxy, propoxy, 2-propoxy, butoxy and tert-butoxy.

The term “haloalkoxy” refers to a haloalkyl group, as defined herein, appended through an oxygen atom. In some embodiments, the alkoxy moiety has 1 to 8 carbon atoms (“C₁₋₈ haloalkoxy”). In some embodiments, the alkoxy moiety has 1 to 6 carbon atoms (“C₁₋₆ haloalkoxy”). In some embodiments, the alkoxy moiety has 1 to 4 carbon atoms (“C₁₋₄ haloalkoxy”). In some embodiments, the alkoxy moiety has 1 to 3 carbon atoms (“C₁₋₃ haloalkoxy”). In some embodiments, the alkoxy moiety has 1 to 2 carbon atoms (“C₁₋₂ haloalkoxy”). Representative examples of haloalkoxy include, but are not limited to, difluoromethoxy, trifluoromethoxy, and 2,2,2-trifluoroethoxy.

The term “alkoxyalkyl” is a substituted alkyl group, wherein one or more of the hydrogen atoms are independently replaced by an alkoxy group, as defined herein. In some embodiments, the alkoxyalkyl moiety has 1 to 8 carbon atoms (“C₁₋₈ alkoxyalkyl”). In some embodiments, the alkoxyalkyl moiety has 1 to 6 carbon atoms (“C₁₋₆ alkoxyalkyl”). In some embodiments, the alkoxyalkyl moiety has 1 to 4 carbon atoms (“C₁₋₄ alkoxyalkyl”). In some embodiments, the alkoxyalkyl moiety has 1 to 3 carbon atoms (“C₁₋₃ alkoxyalkyl”). In some embodiments, the alkoxyalkyl moiety has 1 to 2 carbon atoms (“C₁₋₂ alkoxyalkyl”). By way of example, a C₃alkoxyC₃alkyl group includes, but is not limited to, the groups having the formula:

—CH₂CH₂CH₂OCH₂CH₂CH₃, —CH₂CH₂CH₂OCH(CH₃)₂, —CH(CH₃)CH₂OCH(CH₃)₂,

The term “heteroalkyl” refers to an alkyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. By way of example, a heteroC₁₋₆alkyl (which may also be designated a C₁₋₆heteroalkyl) group includes, but is not limited to, the following structures:

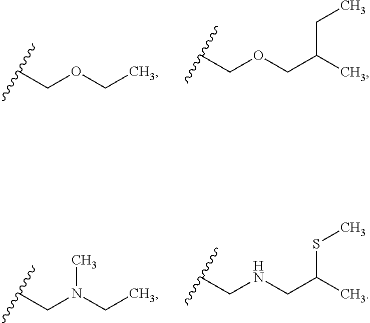

The term “heteroalkyl” preceded by a separate heteroatom refers to a heteroalkyl group bonded through the specified heteroatom. By way of example, a OC₁₋₆heteroalkyl group includes, but it not limited to, the following structures:

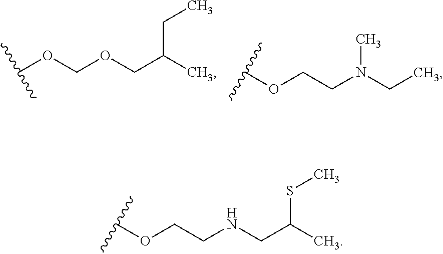

In certain embodiments, a heteroalkyl group refers to a saturated group having from 1 to 20 carbon atoms and 1 or more heteroatoms within the parent chain (“heteroC₁₋₂₀ alkyl”). In some embodiments, a heteroalkyl group is a saturated group having 1 to 18 carbon atoms and lor more heteroatoms within the parent chain (“heteroC₁₋₁₈ alkyl”). In some embodiments, a heteroalkyl group is a saturated group having 1 to 16 carbon atoms and/or more heteroatoms within the parent chain (“heteroC₁₋₁₆ alkyl”). In some embodiments, a heteroalkyl group is a saturated group having 1 to 14 carbon atoms and 1 or more heteroatoms within the parent chain (“heteroC₁₋₁₄ alkyl”). In some embodiments, a heteroalkyl group is a saturated group having 1 to 12 carbon atoms and 1 or more heteroatoms within the parent chain (“heteroC₁₋₁₂ alkyl”). In some embodiments, a heteroalkyl group is a saturated group having 1 to 10 carbon atoms and lor more heteroatoms within the parent chain (“heteroC₁₋₁₀ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃alkyl”). In some embodiments, a heteroalkyl group is a saturated group having 1 to 2 carbon atoms and 1 heteroatom within the parent chain (“heteroC₁₋₂alkyl”). In some embodiments, a heteroalkyl group is a saturated group having 1 carbon atom and 1 heteroatom (“heteroC₁alkyl”). In some embodiments, the heteroalkyl group defined herein is a partially unsaturated group having 1 or more heteroatoms within the parent chain and at least one unsaturated carbon, such as a carbonyl group. For example, a heteroalkyl group may comprise an amide or ester functionality in its parent chain such that one or more carbon atoms are unsaturated carbonyl groups. Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₂₀alkyl. In certain embodiments, the heteroalkyl group is an unsubstituted heteroC₁₋₁₀alkyl. In certain embodiments, the heteroalkyl group is a substituted heteroC₁₋₂₀alkyl. In certain embodiments, the heteroalkyl group is an unsubstituted heteroC₁₋₁₀alkyl.

The term “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆alkenyl”). In some embodiments, an alkenyl group has 2 to 5 carbon atoms (“C₂₋₅alkenyl”). In some embodiments, an alkenyl group has 2 to 4 carbon atoms (“C₂₋₄alkenyl”). In some embodiments, an alkenyl group has 2 to 3 carbon atoms (“C₂₋₃alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl. In an alkenyl group, a C═C double bond for which the stereochemistry is not specified

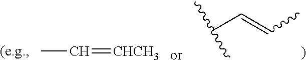

may be an (E)- or (Z)-double bond.

The term “heteroalkenyl” refers to an alkenyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀alkenyl”). In some embodiments, a heteroalkenyl group has 2 to 9 carbon atoms at least one double bond, and 1 or more heteroatoms within the parent chain (“heteroC₂₋₉alkenyl”).

In some embodiments, a heteroalkenyl group has 2 to 8 carbon atoms, at least one double bond, and 1 or more heteroatoms within the parent chain (“heteroC₂₋₈alkenyl”). In some embodiments, a heteroalkenyl group has 2 to 7 carbon atoms, at least one double bond, and 1 or more heteroatoms within the parent chain (“heteroC₂₋₇alkenyl”). In some embodiments, a heteroalkenyl group has 2 to 6 carbon atoms, at least one double bond, and 1 or more heteroatoms within the parent chain (“heteroC₂₋₆alkenyl”). In some embodiments, a heteroalkenyl group has 2 to 5 carbon atoms, at least one double bond, and 1 or 2 heteroatoms within the parent chain (“heteroC₂₋₅alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃alkenyl”). In some embodiments, a heteroalkenyl group has 2 to 6 carbon atoms, at least one double bond, and 1 or 2 heteroatoms within the parent chain (“heteroC₂₋₆alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀alkenyl. In certain embodiments, the heteroalkenyl group is a substituted heteroC₂₋₁₀alkenyl.

The term “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀alkynyl”). In some embodiments, an alkynyl group has 2 to 9 carbon atoms (“C₂₋₉alkynyl”). In some embodiments, an alkynyl group has 2 to 8 carbon atoms (“C₂₋₈alkynyl”). In some embodiments, an alkynyl group has 2 to 7 carbon atoms (“C₂₋₇alkynyl”). In some embodiments, an alkynyl group has 2 to 6 carbon atoms (“C₂₋₆alkynyl”). In some embodiments, an alkynyl group has 2 to 5 carbon atoms (“C₂₋₅alkynyl”). In some embodiments, an alkynyl group has 2 to 4 carbon atoms (“C₂₋₄alkynyl”). In some embodiments, an alkynyl group has 2 to 3 carbon atoms (“C₂₋₃alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀alkynyl”). In some embodiments, a heteroalkynyl group has 2 to 9 carbon atoms, at least one triple bond, and lor more heteroatoms within the parent chain (“heteroC₂₋₉alkynyl”). In some embodiments, a heteroalkynyl group has 2 to 8 carbon atoms, at least one triple bond, and lor more heteroatoms within the parent chain (“heteroC₂₋₈alkynyl”). In some embodiments, a heteroalkynyl group has 2 to 7 carbon atoms, at least one triple bond, and 1 or more heteroatoms within the parent chain (“heteroC₂₋₇alkynyl”). In some embodiments, a heteroalkynyl group has 2 to 6 carbon atoms, at least one triple bond, and 1 or more heteroatoms within the parent chain (“heteroC₂₋₆alkynyl”). In some embodiments, a heteroalkynyl group has 2 to 5 carbon atoms, at least one triple bond, and 1 or 2 heteroatoms within the parent chain (“heteroC₂₋₅alkynyl”). In some embodiments, a heteroalkynyl group has 2 to 4 carbon atoms, at least one triple bond, and 1 or 2 heteroatoms within the parent chain (“heteroC₂₋₄alkynyl”). In some embodiments, a heteroalkynyl group has 2 to 3 carbon atoms, at least one triple bond, and 1 heteroatom within the parent chain (“heteroC₂₋₃alkynyl”). In some embodiments, a heteroalkynyl group has 2 to 6 carbon atoms, at least one triple bond, and 1 or 2 heteroatoms within the parent chain (“heteroC₂₋₆alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀alkynyl. In certain embodiments, the heteroalkynyl group is a substituted heteroC₂₋₁₀alkynyl.

The term “carbocyclyl,” “cycloalkyl,” or “carbocyclic” refers to a radical of a non-aromatic cyclic hydrocarbon group having from 3 to 14 ring carbon atoms (“C₃₋₁₄carbocyclyl”) and zero heteroatoms in the non-aromatic ring system. In some embodiments, a carbocyclyl group has 3 to 10 ring carbon atoms (“C₃₋₁₀carbocyclyl”). In some embodiments, a carbocyclyl group has 3 to 8 ring carbon atoms (“C₃₋₈carbocyclyl”). In some embodiments, a carbocyclyl group has 3 to 7 ring carbon atoms (“C₃₋₇carbocyclyl”). In some embodiments, a carbocyclyl group has 3 to 6 ring carbon atoms (“C₃₋₆carbocyclyl”). In some embodiments, a carbocyclyl group has 4 to 6 ring carbon atoms (“C₄₋₆carbocyclyl”). In some embodiments, a carbocyclyl group has 5 to 6 ring carbon atoms (“C₅₋₆carbocyclyl”). In some embodiments, a carbocyclyl group has 5 to 10 ring carbon atoms (“C₅₋₁₀carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like.

Exemplary C₃₋₈carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₆), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl

(C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄cycloalkyl”). In some embodiments, a cycloalkyl group has 3 to 10 ring carbon atoms (“C₃₋₁₀cycloalkyl”). In some embodiments, a cycloalkyl group has 3 to 8 ring carbon atoms (“C₃₋₈cycloalkyl”). In some embodiments, a cycloalkyl group has 3 to 6 ring carbon atoms (“C₃₋₆cycloalkyl”). In some embodiments, a cycloalkyl group has 4 to 6 ring carbon atoms (“C₄₋₆cycloalkyl”). In some embodiments, a cycloalkyl group has 5 to 6 ring carbon atoms (“C₅₋₆cycloalkyl”). In some embodiments, a cycloalkyl group has 5 to 10 ring carbon atoms (“C₅₋₁₀cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

As used herein, the term “heterocyclyl” refers to an aromatic (also referred to as a heteroaryl), unsaturated, or saturated cyclic hydrocarbon that includes at least one heteroatom in the cycle. For example, the term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, aziridinyl, oxiranyl, and thiiranyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄aryl”). In some embodiments, an aryl group has 6 ring carbon atoms (“C₆aryl”; e.g., phenyl). In some embodiments, an aryl group has 10 ring carbon atoms (“C₁₀aryl”; e.g., naphthyl such as 1-naphthyl and 2-naphthyl). In some embodiments, an aryl group has 14 ring carbon atoms (“C₁₄aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl, and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl, and phenazinyl.

In general, the inclusion of the prefix “alk” in front of a substituent name indicates there is an alkyl group (as defined herein) connecting the named substitutent with the rest of the compound. For example, “alkaryl” (which is a subset of alkyl) refers to an alkyl group substituted by an aryl group, wherein the point of attachment is on the alkyl moiety and “alkheteroaryl” (which is a subset of “alkyl”) refers to an alkyl group substituted by a heteroaryl group, wherein the point of attachment is on the alkyl moiety. The number of carbons atoms may be specified in the alkyl chain, the named substituent, or both. For example, C₁₋₂alkC₆aryl refers to a phenyl ring (which may be substituted) connected via a 1-2 carbon alkylene group.

Affixing the suffix “-ene” to a group indicates the group is a polyvalent moiety, e.g., boned to two or more groups. Alkylene is the polyvalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₃, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{, —OSO}₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{c})₂, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀alkyl, C₁₋₁₀perhaloalkyl, C₂₋₁₀ alkenyl, C₂₋₁₀alkynyl, heteroC₁₋₁₀alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb} or ═NOR^{cc}; each instance of R^{aa} is, independently, selected from C₁₋₁₀alkyl, C₁₋₁₀perhaloalkyl, C₂₋₁₀alkenyl, C₂₋₁₀ alkynyl, heteroC₁₋₁₀alkyl, heteroC₂₋₁₀ alkenyl, heteroC₂₋₁₀alkynyl, C₃₋₁₀ carbocyclyl, 3-14 membered heterocyclyl, C₆₋₁₄aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀perhaloalkyl, C₂₋₁₀alkenyl, C₂₋₁₀alkynyl, heteroC₁₋₁₀alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion; each instance of R^{cc} is, independently, selected from hydrogen, C₁₋₁₀alkyl, C₁₋₁₀perhaloalkyl, C₂₋₁₀alkenyl, C₂₋₁₀alkynyl, heteroC₁₋₁₀ alkyl, heteroC₂₋₁₀alkenyl, heteroC₂₋₁₀alkynyl, C₃₋₁₀carbocyclyl, 3-14 membered heterocyclyl, C₆₋₁₄aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion; each instance of R^{ee} is, independently, selected from C₁₋₆alkyl, C₁₋₆perhaloalkyl, C₂₋₆alkenyl, C₂₋₆alkynyl, heteroC₁₋₆ alkyl, heteroC₂₋₆alkenyl, heteroC₂₋₆alkynyl, C₃₋₁₀carbocyclyl, C₆₋₁₀aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(═NH)NH(C₁₋₆ alkyl), —OC(═NH)NH₂, —NHC(═NH)N(C_{f-6} alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂(C₁₋₆ alkyl), —SO₂O(C₁₋₆ alkyl), —OSO₂(C₁₋₆ alkyl), —SO(C₁₋₆ alkyl), —Si(C₁₋₆ alkyl)₃, —OSi(C₁₋₆ alkyl)₃, —C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆alkyl, C₁₋₆perhaloalkyl, C₂₋₆alkenyl, C₂₋₆alkynyl, heteroC₁₋₆alkyl, heteroC₂₋₆alkenyl, heteroC₂₋₆alkynyl, C₁₋₁₀carbocyclyl, C₆₋₁₀aryl, 3-10 membered heterocyclyl, 5-10 membered heteroaryl; or two geminal R^{gg} substituents can be joined to form ═O or ═S; wherein X⁻ is a counterion.

The term “halo” or “halogen” refers to fluorine (fluoro, —F), chlorine (chloro, —Cl), bromine (bromo, —Br), or iodine (iodo, —I).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, —C(═S)O(R^{X1}), —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphatiethioxy, alkylthioxy, heteroalkylthioxy, arylthioxy, heteroarylthioxy, mono- or di-aliphaticamino, mono- or di-heteroaliphaticamino, mono- or dialkylamino, mono- or di-heteroalkylamino, mono- or di-arylamino, or mono- or diheteroarylamino; or two R^{X1} groups taken together form a 5- to 6-membered heterocyclic ring.

Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers a group wherein the carbon directly attached to the parent molecule is sp² hybridized, and is substituted with an oxygen, nitrogen or sulfur atom, e.g., a group selected from ketones (e.g., —C(═O)R^{aa}), carboxylic acids (e.g., —CO₂H), aldehydes(CHO), esters (e.g., —CO₂R^{aa}, —C(═O)SR^{aa}, —C(═S)SR^{aa}), amides (e.g., —C(═O)N(R^{bb})₂, C(═O)NR^{bb}SO₂R^{aa}, —C(═S)N(R^{bb})₂, and imines (e.g., —C(═NR^{bb})R^{aa}, —C(═NR^{bb})OR^{aa}), C(═NR^{bb})N(R^{bb})₂, wherein R^{aa} and R^{bb} are as defined herein.

The term “oxo” refers to the group ═O, and the term “thiooxo” refers to the group ═S.

The term “cyano” refers to the group —CN.

The term “azide” and “azido” refers to the group —N₃.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or a 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined herein.

As used herein, the designation of a polyvalent moiety without specifying the specific order of attachment is intended to cover all possible arrangements. By way of example, a compound represented by the formula:

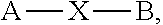

wherein X is NHC(═O) embraces both:

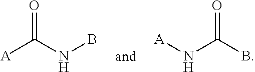

As used herein, a chemical bond depicted: [symbol] represents either a single, double, or triple bond, valency permitting. By way of example,

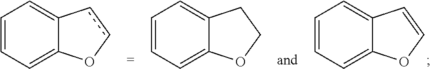

An electron-withdrawing group is a functional group or atom that pulls electron density towards itself, away from other portions of the molecule, e.g., through resonance and/or inductive effects. Exemplary electron-withdrawing groups include F, Cl, Br, I, NO₂, CN, SO₂R, SO₃R, SO₂NR₂, C(O)R^{1a}; C(O)OR, and C(O)NR₂ (wherein R is H or an alkyl, aryl, heteroaryl, cycloalkyl, heterocyclyl group) as well as alkyl group substituted with one or more of those group

An electron-donating group is a functional group or atom that pushes electron density away from itself, towards other portions of the molecule, e.g., through resonance and/or inductive effects. Exemplary electron-donating groups include unsubstituted alkyl or aryl groups, OR and N(R)₂ and alkyl groups substituted with one or more OR and N(R)₂ groups.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture. Unless stated to the contrary, a formula depicting one or more stereochemical features does not exclude the presence of other isomers.

Some compounds disclosed herein may exist as one or more tautomers. Tautomers are interconvertible structural isomers that differ in the position of one or more protons or other labile atom. By way of example:

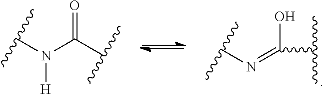

The prevalence of one tautomeric form over another will depend on the specific chemical compound as well as its local chemical environment. Unless specified to the contrary, the depiction of one tautomeric form is inclusive of all possible tautomeric forms.

Unless stated to the contrary, a substituent drawn without explicitly specifying the point of attachment indicates that the substituent may be attached at any possible atom. For example, in a benzofuran depicted as:

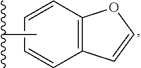

the substituent may be present at any one of the six possible carbon atoms.

As used herein, the term “null,” when referring to a possible identity of a chemical moiety, indicates that the group is absent, and the two adjacent groups are directly bonded to one another. By way of example, for a genus of compounds having the formula CH₃—X—CH₃, if X is null, then the resulting compound has the formula CH₃—CH₃.

Disclosed herein are fluorescent compounds having the formula:

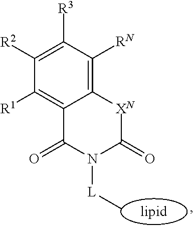

and physiologically acceptable salts thereof, wherein
X^{N} is null or together forms an aromatic ring with R^{N}, wherein when X^{N} is null, R^{N} is selected from F, Cl, Br, I, CN, R^{N*}, N(R^{Na})(R^{Nb}), OR^{Nc}, SO₃R^{N*}, SO₂R^{N*}, SO₂N(R^{N*})₂, C(O)R^{N*}; C(O)OR^{N*}, or OC(O)R^{N*}, wherein R^{N*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{N*} groups can together form a ring;
R^{Na} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{Nb} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{Na} and R^{Nb} can together form a ring;
R^{Nc} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₃₂alkyl;
R¹ is selected from F, Cl, Br, I, CN, R^{1*}, N(R^{1a})(R^{1b}), OR^{1c}, SO₃R^{1*}, SO₂R^{1*}, SO₂N(R^{1*})₂, C(O)R^{1*}; C(O)OR^{1*}, or OC(O)R^{1*}, wherein R^{1*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{1*} groups can together form a ring;
R^{1a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{1b} is selected from hydrogen, C₁₋₈alkyl, C₁₋₈heteroalkyl, aryl, C₁₋₈heteroaryl, C₃₋₈cycloalkyl, or C₁₋₈heterocyclyl;
wherein R^{1a} and R^{1b} can together form a ring;
R^{1c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₃₂alkyl;
R² is selected from F, Cl, Br, I, CN, R^{2*}, N(R^{2a})(R^{2b}), OR^{2c}, SO₃R^{2*}, SO₂R^{2*}, SO₂N(R^{2*})₂, C(O)R^{2*}; C(O)OR^{2*}, or OC(O)R^{2*}, wherein R^{2*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{2*} groups can together form a ring;
R^{2a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{2b} is selected from hydrogen, C₁₋₈alkyl, C₁₋₈heteroalkyl, aryl, C₁₋₈heteroaryl, C₃₋₈cycloalkyl, or C₁₋₈heterocyclyl;
wherein R^{2a} and R^{2b} can together form a ring;
R^{2c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₃₂alkyl;
R³ is selected N(R^{3a})(R^{3b}) and OR^{3c};
R^{3a} is selected from hydrogen, NH₂, OH, C₁₋₈alkyl, C₁₋₈heteroalkyl, aryl, C₁₋₈heteroaryl, C₃₋₈cycloalkyl, or C₁₋₈heterocyclyl;
R^{3b} is selected from hydrogen, C₁₋₈alkyl, C₁₋₈heteroalkyl, aryl, C₁₋₈heteroaryl, C₃₋₈cycloalkyl, or C₁₋₈heterocyclyl;
wherein R^{3a} and R^{3b} can together form a ring;
R^{3c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₃₂alkyl;
wherein L has the formula:

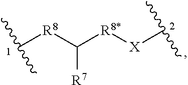

wherein X is null C(═O), OC(═O), NHC(═O);
R⁷ is selected from F, Cl, Br, I, NO₂, CN, R^{7*}, OR^{7*}, N(R^{6a})(R^{6b}), SO₃R^{7*}, SO₂R^{7*}, SO₂N(R^{7*})₂, C(O)R^{7*}; C(O)OR^{7*}, OC(O)R^{7*}; C(O)N(R^{7*})₂, N(R^{7*})C(O)R^{7*}, OC(O)N(R^{7*})₂, N(R^{7*})C(O)N(R^{7*})₂, wherein R^{7*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
R⁸ is selected from null, C₁₋₈alkylene, C₁₋₈heteroalkylene, arylene, C₁₋₈heteroarylene, C₃₋₈cycloalkylene, or C₁₋₈heterocyclylene;
R^{8*} is selected from null, C₁₋₈alkylene, C₁₋₈heteroalkylene, arylene, C₁₋₈heteroarylene, C₃₋₈cycloalkylene, or C₁₋₈heterocyclylene;
wherein any two of R⁷, R⁸, and R^{8*} can together form a ring;
wavy line 1 represents the point of attachment to imide nitrogen, and wave line two represents the point of attachment to the lipid,
wherein when R⁷ is H, then R⁸ and R^{8*} are not both null.

In some implementation, X^{N} is null, which produces a phthalimide having the formula:

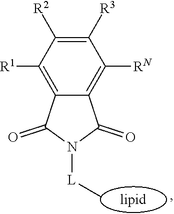

and R¹ and R^{N} are both hydrogen.

In some implementations, R^{N} and X^{N} together form a ring having the formula:

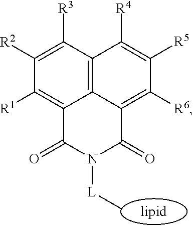

R⁴ is selected from F, Cl, Br, I, CN, R^{4*}, N(R^{1a})(R^{1b}), OR^{1c}, SO₃R^{4*}, SO₂R^{4*}, SO₂N(R^{4*})₂, C(O)R^{4*}; C(O)OR^{4*}, or OC(O)R^{4*}, wherein R^{4*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{4*} groups can together form a ring;
R^{4a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{4b} is selected from hydrogen, C₁₋₈alkyl, C₁₋₈heteroalkyl, aryl, C₁₋₈heteroaryl, C₃₋₈cycloalkyl, or C₁₋₈heterocyclyl;
wherein R^{4a} and R^{4b} can together form a ring;
R^{4c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₃₂alkyl;
R⁵ is selected from F, Cl, Br, I, CN, R^{5*}, N(R^{1a})(R^{1b}), OR^{1c}, SO₃R^{5*}, SO₂R^{5*}, SO₂N(R^{5*})₂, C(O)R^{5*}; C(O)OR^{5*}, or OC(O)R^{5*}, wherein R^{5*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{5*} groups can together form a ring;
R^{5a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{5b} is selected from hydrogen, C₁₋₈alkyl, C₁₋₈heteroalkyl, aryl, C₁₋₈heteroaryl, C₃₋₈cycloalkyl, or C₁₋₈heterocyclyl;
wherein R^{5a} and R^{5b} can together form a ring;
R^{5c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₂₅alkyl;
R⁶ is selected from F, Cl, Br, I, CN, R^{6*}, N(R^{1a})(R^{1b}), OR^{1c}, SO₃R^{6*}, SO₂R^{6*}, SO₂N(R^{6*})₂, C(O)R^{6*}; C(O)OR^{6*}, or OC(O)R^{6*}, wherein R^{6*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{6*} groups can together form a ring;
R^{6a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{6b} is selected from hydrogen, C₁₋₈alkyl, C₁₋₈heteroalkyl, aryl, C₁₋₈heteroaryl, C₃₋₈cycloalkyl, or C₁₋₈heterocyclyl;
wherein R^{6a} and R^{6b} can together form a ring;
R^{6c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₅₋₂₆alkyl;

In some implementations, R¹ and R² together form a phenyl ring, having the formula:

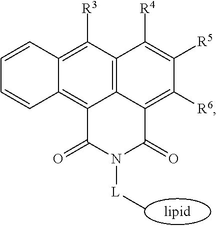

In some implementations, one or more of R³, R⁴, R⁵, and R⁶ can be hydrogen, for example in some implementations R⁵ can be hydrogen. In certain implementations, R⁶ is hydrogen. In some implementations, R⁴ is hydrogen. In some implementations, R³ is hydrogen.

In some implementations, the compound can have the formula:

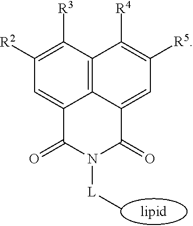

In some implementations, R² and R⁵ are independently selected from H, F, Cl, Br, C₁₋₄ alkyl, OH, OC₁₋₄alkyl, COOH, SO₃H, NH₂, NHCH₃, N(CH₃)₂.

In some implementations, R³ is N(R^{3a})(R^{3b}).

In some implementations, R³has the formula:

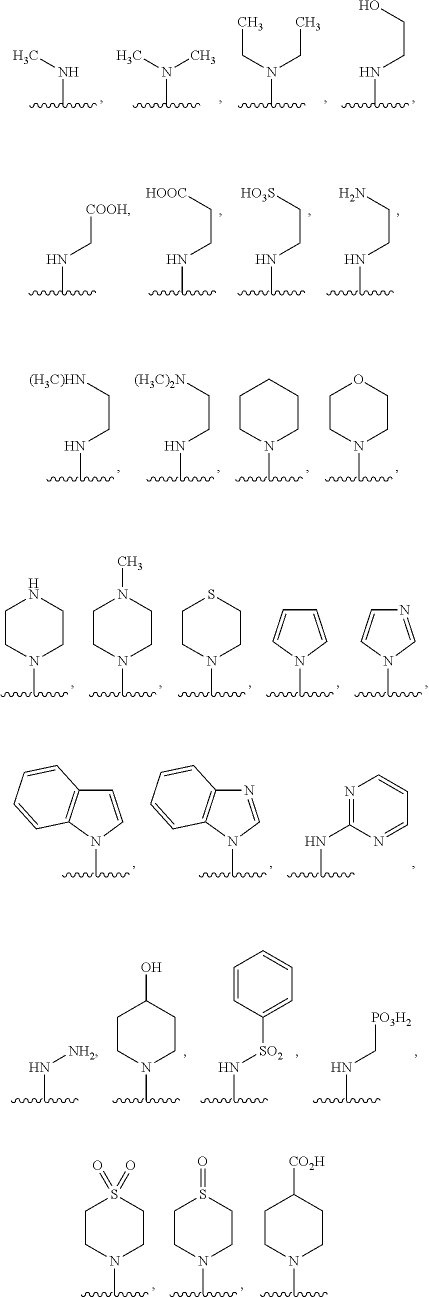

In some implementations, R³ is OC(═O)R^{fa}, wherein OC(═O)R^{fa} is derived from crotonic acid, myristoleic, palmitoleic acid, sapienic acid, oleic acid, elaidic acid, vaccenic acid, gadoleic acid, eicosenoic acid, erucic acid, linoleic acid, eicosadienoic acid, docosadienoic acid, linolenic acid, pinolenic acid, eleostearic acid, mead acid, dihomo-γ-linolenic acid, eicosatrienoic acid, tetra-unsaturated fatty acids, stearidonic acid, arachidonic acid, eicosatetraenoic acid, adrenic acid, bosseopentaenoic acid, eicosapentaenoic acid, ozubondo acid, sardine acid, tetracosanolpentaenoic acid, docosahexaenoic acid, herring acid, petroselinic acid, paullinic acid, gondoic acid, erucic acid, brassidic acid, nervonic acid, arachidonic acid, or a combination thereof.

In some implementations, R^{fa} is a C₈₋₃₂alkyl group, C₈₋₂₄alkyl group, a C₈₋₁₈alkyl group, or C₁₀₋₁₈alkyl group.

In some implementations, R^{fa} is an alkyl group that comprises 1, 2, or 3 olefinic bonds.

In some implementations, R^{fa} is a saturated alkyl group.

In some implementations, L has the formula:

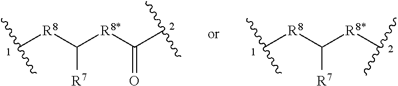

wherein R⁸ is null or C₁₋₈alkylene, R^{8*} is null or C₁₋₈alkylene, and R⁷ is NH₂, OH, SH, or C₁₋₆alkyl.

In some implementations, R⁷ is C₁₋₆alkyl, substituted one or more times by F, Cl, Br, I, CN, NO₂, OR⁹, SR⁹, N(R⁹)₂, COOR⁹, NHC(═NH)N(R⁹)₂, or CON(R⁹)₂, wherein R⁹ is independently selected from hydrogen, C₁₋₈alkyl, C₁₋₈heteroalkyl, aryl, C₁₋₈heteroaryl, C₃₋₈cycloalkyl, or C₁₋₈heterocyclyl.

In some implementations, R⁸ and R^{8*} are null and R⁷ is CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃.

In some implementations, R⁸ and R^{8*} are null and R⁷ is CH₂OH or CH(OH)CH₃.

In some implementations, R⁸ is CH₂, R^{8*} is null, and R⁷ is H, OH, NH₂, SH, COOH, CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃.

In some implementations, R⁸ is CH₂CH₂, R^{8*} is null, and R⁷ is H, OH, NH₂, SH, COOH, CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃.

In some implementations, R⁸ is CH₂CH₂CH₂, R^{8*} is null, and R⁷ is H, OH, NH₂, SH, COOH, CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃.

In some implementations, R⁸ is CH₂CH₂CH₂CH₂, R^{8*} is null, and R⁷ is H, OH, NH, SH, COOH, CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃.

In some implementations, L has the formula:

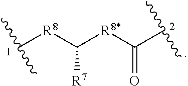

In some implementations, R¹ is H.

In some implementations, R² is H.

In some implementations, R⁵ is H.

In some implementations, R⁶ is H.

In some implementations, the lipid is a sterol, e.g., a four-ring carbocycle having the general formula:

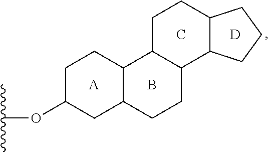

wherein the dashed line indicates the point of attachment to L. Each of the A, B, C, and D rings may be substituted one or more times. Exemplary substituents include alkyl (including saturated alkyl and unsaturated alkyl), oxo, hydroxy, acyl and the like.

In some implementations, the lipid has the formula:

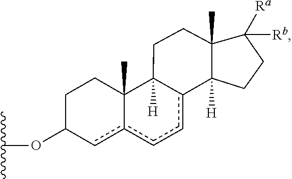

wherein R^{a} is selected from H, OH, or C₁₋₁₂alkyl, and Re is H, or R^{a} and R^{b} together form an oxo, wherein each [symbol] is independently a single or double bond, with the proviso that the compound does not include an allene;

In some implementations, the lipid has the formula:

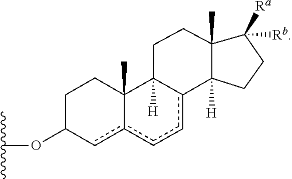

In some implementations, R^{a} is a C₂₋₁₂alkyl group that contains zero, one, or two double bonds.

In some implementations, the lipid has the formula:

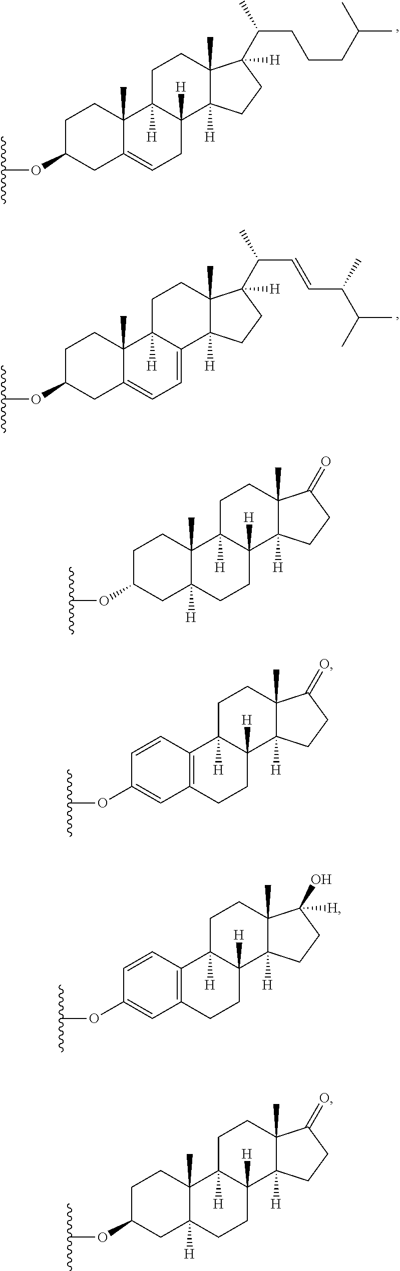

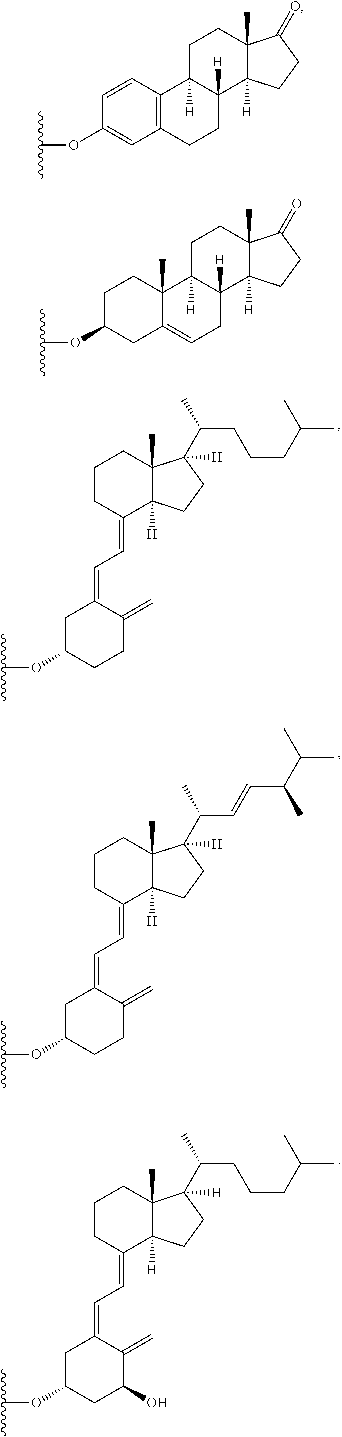

In some implementations, the lipid has the formula:

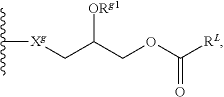

wherein X^{g} is O or NH, and R^{g1} is H or C(═O)R^{L}, wherein OC(═O)R^{L} is derived from crotonic acid, myristoleic, palmitoleic acid, sapienic acid, oleic acid, elaidic acid, vaccenic acid, gadoleic acid, eicosenoic acid, erucic acid, linoleic acid, eicosadienoic acid, docosadienoic acid, linolenic acid, pinolenic acid, eleostearic acid, mead acid, dihomo-γ-linolenic acid, eicosatrienoic acid, tetra-unsaturated fatty acids, stearidonic acid, arachidonic acid, eicosatetraenoic acid, adrenic acid, bosseopentaenoic acid, eicosapentaenoic acid, ozubondo acid, sardine acid, tetracosanolpentaenoic acid, docosahexaenoic acid, herring acid, petroselinic acid, paullinic acid, gondoic acid, erucic acid, brassidic acid, nervonic acid, arachidonic acid, or a combination thereof.

In some implementations, R^{L} is a C₈₋₃₂alkyl group, C₈₋₂₄alkyl group, a C₈₋₁₈alkyl group, or C₁₀₋₁₈alkyl group.

In some implementations, R^{L} is an alkyl group that comprises 1, 2, or 3 olefinic bonds.

In some implementations, R^{L} is a saturated alkyl group.

Also disclosed are methods of imaging or manipulating a biological system, including the step of contacting the biological system with one or more of the disclosed compounds irradiating the compound. The irradiation can be at a wavelength of 300-700 nm, at 300-600 nm, at 300-500 nm, at 300-400 nm, at 350-450 nm, at 400-500 nm, at 450-500 nm, at 500-600 nm, or at 550-650 nm. In some implementations the biological systems can include an animal, organ, cell, organelles, cell membrane, and molecular complexes containing lipids.

The compounds of the disclosure may be prepared by condensation of a lipid with a suitably protected amino acid:

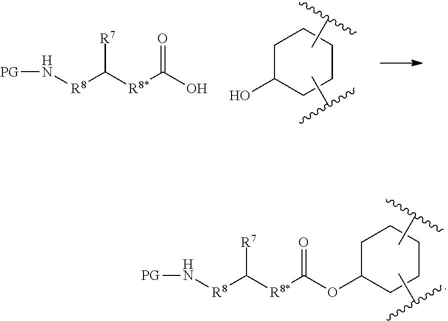

Following deprotection of the nitrogen atom, the amine may be reacted with a suitably functionalized anhydride:

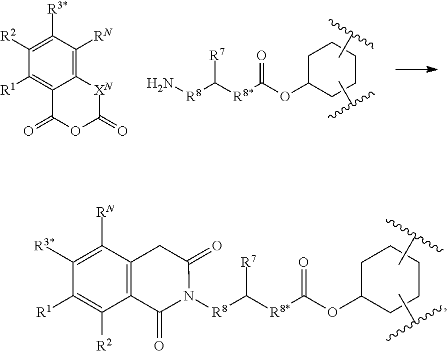

wherein R^{3*} is R³ or a leaving group like halogen (e.g., F, Cl, Br, I), tosylate, mesylate, triflate, etc. When R^{3*} is a leaving group, the compound may be combined with an appropriate headgroup nucleophile (e.g., HN(R^{3a})(R^{3b}) or HOR^{3c}) to provide the compounds of the disclosure.

According to these processes, the following compounds were prepared and evaluated:

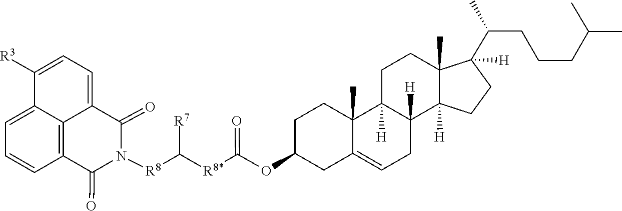

| Ex. | R⁸ | R⁷ | R^{8*} | R³ |
|---|---|---|---|---|
| CND1 | null | H | null | 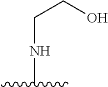 [SMILES: *NCCO] |
| CND2 | null | H | null | 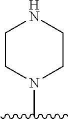 [SMILES: *N1CCNCC1] |
| CND3 | null | CH₂OH | null | 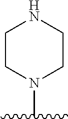 [SMILES: *N1CCNCC1] |
| CND4 | CH₂ | H | null | 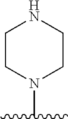 [SMILES: *N1CCNCC1] |
| CND5 | null | H | null | 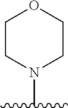 [SMILES: *N1CCOCC1] |
| CND6 | null | CH₂OH | null | 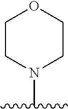 [SMILES: *N1CCOCC1] |
| CND7 | null | H | null | 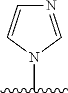 [SMILES: *n1ccnc1] |
| CND8 | null | H | null | —OH |
| CND9 | null | H | null | 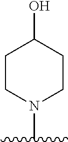 [SMILES: *N1CCC(O)CC1] |
| CND10 | null | H | null | 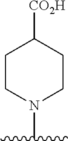 [SMILES: *N1CCC(C(=O)O)CC1] |

Data for the prepared compounds is reported in the attached figures.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A compound having the formula: 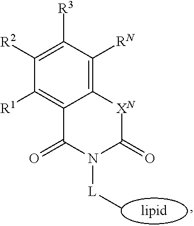
or a physiologically acceptable salt thereof, wherein:
X^{N} is null or together forms an aromatic ring with R^{N};
wherein when X^{N} is null, R^{N} is selected from F, Cl, Br, I, CN, R^{N*}, N(R^{Na})(R^{Nb}), OR^{Nc}, SO₃R^{N*}, SO₂R^{N*}, SO₂N(R^{N*})₂, C(O)R^{N*}, C(O)OR^{N*}, or OC(O)R^{N*};
wherein R^{N*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{N*} groups can together form a ring;
R^{Na} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{Nb} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{Na} and R^{Nb} can together form a ring;
R^{Nc} is selected from hydrogen, C₁₋₈alkyl, C₁₋₈heteroalkyl, aryl, C₁₋₈heteroaryl, C₃₋₈cycloalkyl, C₁₋₈ heterocyclyl, or C(═O)C₈₋₃₂alkyl;
R¹ is selected from F, Cl, Br, I, CN, R^{1*}, N(R^{1a})(R^{1b}), OR^{1c}, SO₃R^{1*}, SO₂R^{1*}, SO₂N(R^{1*})₂, C(O)R^{1*}, C(O)OR^{1*}, or OC(O)R^{1*};
wherein R^{1*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{1*} groups can together form a ring;
R^{1a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{1b} is selected from hydrogen, C₁₋₈alkyl, C₁₋₈heteroalkyl, aryl, C₁₋₈heteroaryl, C₃₋₈cycloalkyl, or C₁₋₈heterocyclyl;
in R^{1a} and R^{1b} can together form a ring;
R^{1c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₃₂alkyl;
R² is selected from F, Cl, Br, I, CN, R^{2*}, N(R^{2a})(R^{2b}), OR^{2c}, SO₃R^{2*}, SO₂R^{2*}, SO₂N(R^{2*})₂, C(O)R^{2*}, C(O)OR^{2*}, or OC(O)R^{2*};
wherein R^{2*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{2*} groups can together form a ring;
R^{2a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{2b} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{2a} and R^{2b} can together form a ring;
R^{2c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₃₂alkyl;
R³ is selected from N(R^{3a})(R^{3b}) and OR^{3c};
R^{3a} is selected from hydrogen, NH₂, OH, C₁₋₈alkyl, C₁₋₈heteroalkyl, aryl, C₁₋₈heteroaryl, C₃₋₈cycloalkyl, or C₁₋₈heterocyclyl;
R^{3b} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{3a} and R^{3b} can together form a ring;
R^{3c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₃₂alkyl;
L has the formula: 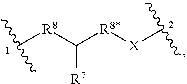
wherein X is null C(═O), OC(═O), or NHC(═O);
R⁷ is selected from F, Cl, Br, I, NO₂, CN, R^{7*}, OR^{7*}, N(R^{6a})(R^{6b}), SO₃R^{7*}, SO₂R^{7*}, SO₂N(R^{7*})₂, C(O)R^{7*}, C(O)OR^{7*}, OC(O)R^{7*}, C(O)N(R^{7*})₂, N(R^{7*})C(O)R^{7*}, OC(O)N(R^{7*})₂, N(R^{7*})C(O)N(R^{7*})₂;
wherein R^{7*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
R⁸ is selected from null, C₁₋₈alkylene, C₁₋₈heteroalkylene, arylene, C₁₋₈heteroarylene, C₃₋₈cycloalkylene, or C₁₋₈heterocyclylene;
R^{8*} is selected from null, C₁₋₈alkylene, C₁₋₈heteroalkylene, arylene, C₁₋₈heteroarylene, C₃₋₈cycloalkylene, or C₁₋₈heterocyclylene;
any two of R⁷, R⁸, and R^{8*} can together form a ring;
wavy line 1 represents the point of attachment to imide nitrogen, and wavy line two represents the point of attachment to the lipid; and
when R⁷ is H, then R⁸ and R^{8*} are not both null.

2. The compound according to claim 1, wherein X^{N} is null, and
having the formula: 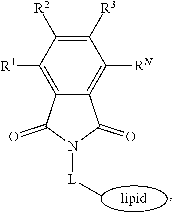
wherein R¹ and R^{N} are both hydrogen.

3. The compound according to claim 1, wherein R^{N} and X^{N} together form a ring, and having the formula: 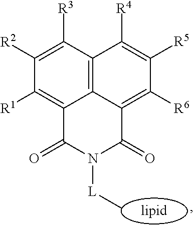
wherein:
R⁴ is selected from F, Cl, Br, I, CN, R^{4*}, N(R^{1a})(R^{1b}), OR^{1c}, SO₃R^{4*}, SO₂R^{4*}, SO₂N(R^{4*})₂, C(O)R⁴*, C(O)OR^{4*}, or OC(O)R^{4*};
wherein R^{4*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{4*} groups can together form a ring;
R^{4a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{4b} is selected from hydrogen, C₁₋₈alkyl, C₁₋₈heteroalkyl, aryl, C₁₋₈heteroaryl, C₃₋₈cycloalkyl, or C₁₋₈heterocyclyl;
in R^{4a} and R^{4b} can together form a ring;
R^{4c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₃₂alkyl;
R⁵ is selected from F, Cl, Br, I, CN, R^{5*}, N(R^{1a})(R^{1b}), OR^{1c}, SO₃R^{5*}, SO₂R^{5*}, SO₂N(R^{5*})₂, C(O)R^{5*}, C(O)OR^{5*}, or OC(O)R^{5*};
wherein R^{5*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{5*} groups can together form a ring;
R^{5a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{5b} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{5a} and R^{5b} can together form a ring;
R^{5c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₂₅alkyl;
R⁶ is selected from F, Cl, Br, I, CN, R^{6*}, N(R^{1a})(R^{1b}), OR^{1c}, SO₃R^{6*}, SO₂R^{6*}, SO₂N(R^{6*})₂, C(O)R⁶*% C(O)OR^{6*}, or OC(O)R⁶*;
wherein R^{6*} is in each case independently selected from hydrogen, C₁₋₈alkyl, C₁₋₈heteroalkyl, aryl, C₁₋₈heteroaryl, C₃₋₈cycloalkyl, or C₁₋₈heterocyclyl;
wherein two R^{6*} groups can together form a ring;
R^{6a} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{6b} is selected from hydrogen, C₁₋₈alkyl, C₁₋₈heteroalkyl, aryl, C₁₋₈heteroaryl, C₃₋₈cycloalkyl, or C₁₋₈heterocyclyl;
R^{6a} and R^{6b} can together form a ring; and
R^{6c} is selected from hydrogen, C₁₋₈alkyl, C₁₋₈heteroalkyl, aryl, C₁₋₈heteroaryl, C₃₋₈cycloalkyl, C₁₋₈heterocyclyl, or C(═O)C₈₋₂₆alkyl.

4. The compound according to claim 1, wherein R¹ and R² together form a phenyl ring, and having the formula:

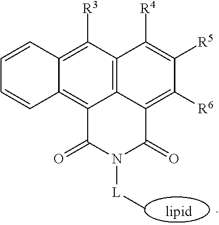

5. (canceled)

6. (canceled)

7. (canceled)

8. (canceled)

9. The compound according to claim 3, having the formula:

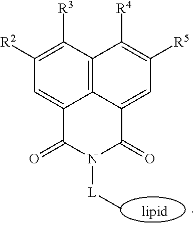

10. The compound according to claim 9, wherein R² and R⁵ are independently selected from F, Cl, Br, C₁₋₄alkyl, OH, OC₁₋₄alkyl, COOH, SO₃H, NH₂, NHCH₃, and N(CH₃)₂; and/or wherein R³ is N(R^{3a})(R^{3b}).

11. (canceled)

12. The compound according to claim 10, wherein R³ has the formula:

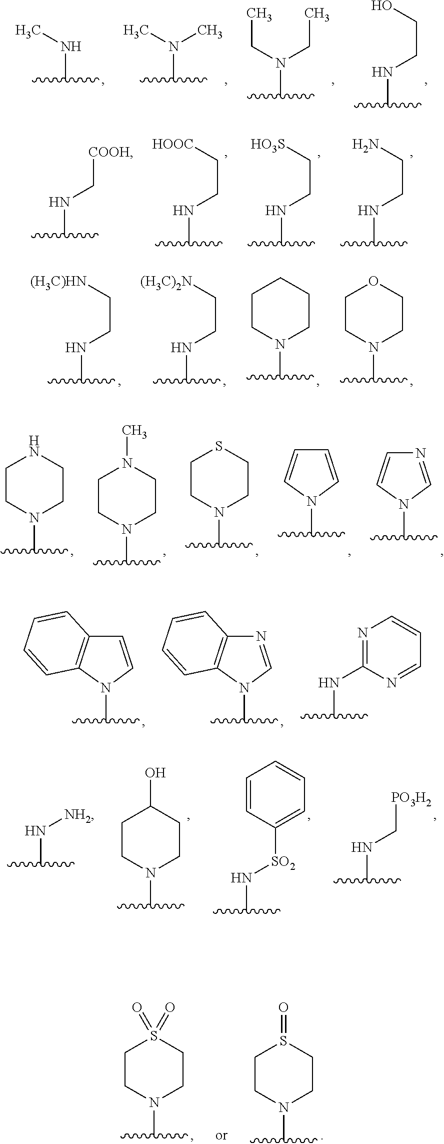

13. The compound according to claim 1, wherein R³ is OC(═O)R^{fa}; and wherein OC(═O)R^{fa} is derived from crotonic acid, myristoleic, palmitoleic acid, sapienic acid, oleic acid, elaidic acid, vaccenic acid, gadoleic acid, eicosenoic acid, erucic acid, linoleic acid, eicosadienoic acid, docosadienoic acid, linolenic acid, pinolenic acid, eleostearic acid, mead acid, dihomo-γ-linolenic acid, eicosatrienoic acid, tetra-unsaturated fatty acids, stearidonic acid, arachidonic acid, eicosatetraenoic acid, adrenic acid, bosseopentaenoic acid, eicosapentaenoic acid, ozubondo acid, sardine acid, tetracosanolpentaenoic acid, docosahexaenoic acid, herring acid, petroselinic acid, paullinic acid, gondoic acid, erucic acid, brassidic acid, nervonic acid, arachidonic acid, or a combination thereof.

14. The compound according to claim 13, wherein R^{fa} is a C₈₋₃₂alkyl group, C₈₋₂₄alkyl group, C₈₋₁₈alkyl group, or C₁₀₋₁₈alkyl group; wherein R^{fa} is an alkyl group that comprises 1, 2, or 3 olefinic bonds; or wherein R^{fa} is a saturated alkyl group.

15. (canceled)

16. (canceled)

17. The compound according to claim 1, wherein L has the formula: 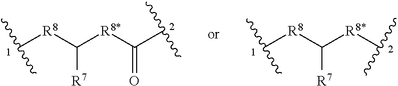
wherein R⁸ is null or C₁₋₈alkylene, R^{8*} is null or C₁₋₈alkylene, and R⁷ is NH₂, OH, SH, or C₁₋₆alkyl.

18. The compound according to claim 17, wherein R⁷ is C₁₋₆alkyl, substituted one or more times by F, Cl, Br, I, CN, NO₂, OR⁹, SR⁹, N(R⁹)₂, COOR⁹, NHC(═NH)N(R⁹)₂, or CON(R⁹)₂; wherein R⁹ is independently selected from hydrogen, C₁₋₈alkyl, C₁₋₈heteroalkyl, aryl, C₁₋₈heteroaryl, C₃₋₈cycloalkyl, or C₁₋₈heterocyclyl.

19. The compound according to claim 17, wherein;
R⁸ and R^{8*} are null and R⁷ is CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃;
R⁸ and R^{8*} are null and R⁷ is CH₂OH or CH(OH)CH₃;
R⁸ is CH₂, R^{8*} is null, and R⁷ is H, OH, NH₂, SH, COOH, CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃;
R⁸ is CH₂CH₂, R^{8*} is null, and R⁷ is H, OH, NH₂, SH, COOH, CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃;
R⁸ is CH₂CH₂CH₂, R^{8*} is null, and R⁷ is H, OH, NH₂, SH, COOH, CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃; or
R⁸ is CH₂CH₂CH₂CH₂, R^{8*} is null, and R⁷ is H, OH, NH₂, SH, COOH, CH₂NH₂, CH₂OH, CH₂SH, CH₂aryl, CH₂heteroaryl, CH(OH)CH₃, CH₂COOH, CH₂CH₂COOH, CH₂CH₂CH₂CH₂NH₂, CH(CH₃)₂, CH₃, CH(CH₃)CH₂CH₃, CH₂CH₂CH(CH₃)₂, or CH₂CH₂SCH₃.

20. (canceled)

21. (canceled)

22. (canceled)

23. (canceled)

24. (canceled)

25. The compound according to claim 17, wherein L has the formula:

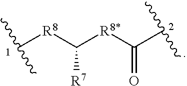

26. (canceled)

27. (canceled)

28. (canceled)

29. (canceled)

30. The compound according to claim 1, wherein the lipid has the formula: 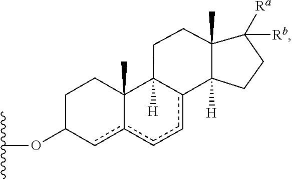
wherein R^{a} is selected from H, OH, or C₁₋₁₂alkyl, and R^{b} is H, or R^{a} and R^{b} together form an oxo.

31. The compound according to claim 30, wherein the lipid has the formula:

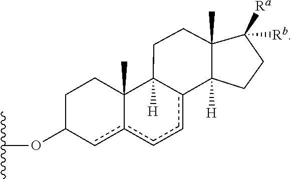

32. (canceled)

33. The compound according to claim 30, wherein the lipid has the formula:

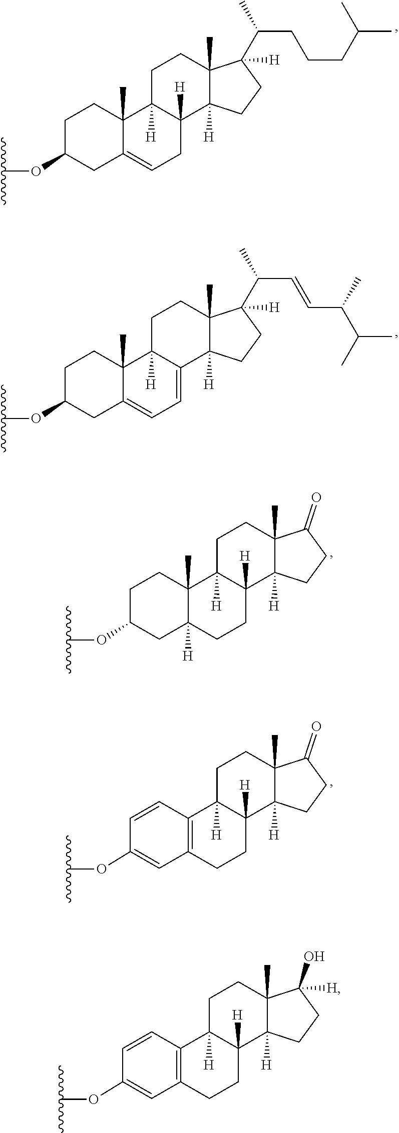

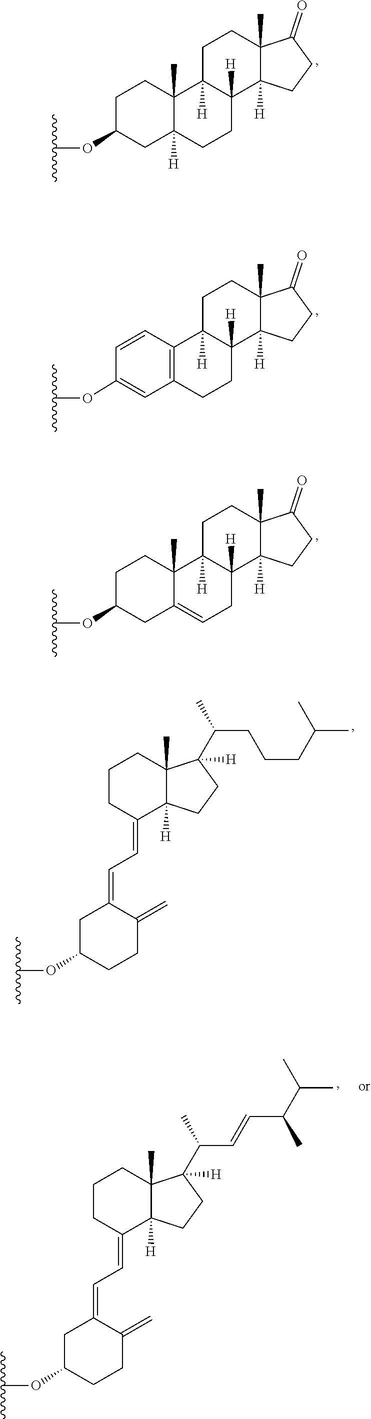

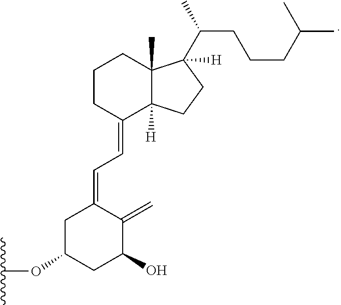 [SMILES: *O[C@@H]1C/C(=C/C=C2\CCC[C@]3(C)[C@@H]([C@H](C)CCCC(C)C)CC[C@@H]23)C(=C)[C@@H](O)C1 ; "."]

34. The compound according to claim 1, wherein lipid has the formula: 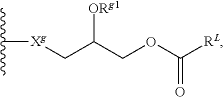
wherein X^{g} is O or NH, and R¹ is H or C(═O)R^{L}; and wherein OC(═O)R^{L} is derived from crotonic acid, myristoleic, palmitoleic acid, sapienic acid, oleic acid, elaidic acid, vaccenic acid, gadoleic acid, eicosenoic acid, erucic acid, linoleic acid, eicosadienoic acid, docosadienoic acid, linolenic acid, pinolenic acid, eleostearic acid, mead acid, dihomo-γ-linolenic acid, eicosatrienoic acid, tetra-unsaturated fatty acids, stearidonic acid, arachidonic acid, eicosatetraenoic acid, adrenic acid, bosseopentaenoic acid, eicosapentaenoic acid, ozubondo acid, sardine acid, tetracosanolpentaenoic acid, docosahexaenoic acid, herring acid, petroselinic acid, paullinic acid, gondoic acid, erucic acid, brassidic acid, nervonic acid, arachidonic acid, or a combination thereof.

35. The compound according to claim 34, wherein R^{L} is a C₈₋₃₂alkyl group, C₈₋₂₄alkyl group, C₈₋₁₈alkyl group, or C₁₀₋₁₈alkyl group; wherein R^{L} is an alkyl group that comprises 1, 2, or 3 olefinic bonds; or wherein R^{L} is a saturated alkyl group.

36. (canceled)

37. (canceled)

38. A method of imaging or manipulating a biological system, the method comprising contacting the biological system with the compound according to claim 1 and irradiating the compound.

39. The method according claim 38, wherein the irradiating comprises irradiating at 300-700 nm; and wherein the biological system comprises an animal, organ, cell, organelles, cell membrane, or molecular complexes containing lipids.

40. (canceled)
